(19) United States
(12) Patent Application Publication
(10) Pub. No.: US 2024/0288451 A1
(43) Pub. Date: Aug. 29, 2024

(54) Chromogenic Absorbent Material for Animal Litter

(71) Applicant: 7905122 CANADA INC., Boucherville (CA)

(72) Inventors: John Manioudakis, Boucherville (CA); Kim Beauregard, Boucherville (CA); Paul Jollez, Boucherville (CA)

(21) Appl. No.: 18/562,048

(22) PCT Filed: May 18, 2022
(86) PCT No.: PCT/CA2022/050782

§ 371 Date: Nov. 17, 2023

Related U.S. Application Data
(60) Provisional application No. 63/190,949, filed on May 20, 2021.

(51) Int. Cl. G01N 33/72; C12Q 1/26; C12Q 1/28; C12Q 1/54; G01N 33/52

(52) U.S. Cl. CPC G01N 33/725; C12Q 1/26; C12Q 1/28; C12Q 1/54; G01N 33/528

(57) ABSTRACT

A chromogenic absorbent material for hemoglobin and glucose detection in an animal excretion is provided. The chromogenic absorbent material comprises a benzidine-type compound, an organic hydroperoxide, an oxidoreductase, a peroxidase, a pseudoperoxidase or a combination thereof, and a polysaccharide matrix, comprising: from about 10 wt % to about 32.5 wt % chemically or mechanically processed non-functionalized cellulose; from about 25 wt % to about 70 wt % pregelatinized starch (PGS); from 0 wt % to about 20 wt % guar gum; from 0 wt % to about 25 wt % methyl hydroxyethyl cellulose (MHEC); from 0 wt % to about 15 wt % hydroxyethyl cellulose (HEC); and from 0 wt % to about 10 wt % carboxymethyl cellulose (CMC).

### CROSS-REFERENCE TO RELATED APPLICATIONS

This application is the national phase of, and claims priority to, International Application No. PCT/CA2022/050782, filed May 18, 2022, which claims priority to U.S. Provisional Patent Application No. 63/190,949, filed May 20, 2021.

### FIELD

The technical field generally relates to chromogenic absorbent materials, and more particularly relates to chromogenic absorbent materials for detection of blood and/or glucose in an animal excretion.

### BACKGROUND

Feline urinary tract disease can be a serious condition for cats. In feline urinary tract disease, crystals of magnesium ammonium phosphate can precipitate in the cat's urinary tract and cause obstruction. If untreated, the obstruction can lead to intense pain and can often be fatal within days. In some cases, upon observing feline urinary tract disease symptoms-such as bloody urine and urination discomfort and straining-cat owners often consult their veterinarian who may be able to provide treatments, which may be expensive. However, many cats with feline urinary tract disease do not show any obvious symptoms, which is why this disease has been referred to as a “silent killer”.

Another serious condition for cats is diabetes. Diabetes strikes about 1 in 400 cats and has become increasingly common. Symptoms of diabetes in cats are similar to those in humans, and about 80% to 95% of diabetic cats experience something similar to type-2 diabetes in humans. Cats suffering of diabetes usually become severely insulin-dependent by the time symptoms are diagnosed. In cats suffering from type-2 diabetes, early treatment can sometimes lead to diabetic remission, in which the cat no longer needs injected insulin. If left untreated, the condition leads to increasingly weak cats, malnutrition, ketoacidosis and/or dehydration, and eventually death.

Early detection of diseases or conditions in animals or humans is therefore of paramount importance in facilitating treatment, lessening the likelihood of severe complications or aggravations, and reducing the cost of treatment.

Several chromogenic absorbent materials are known for use in animal litters to detect blood and/or glucose in animal excretions. However, challenges still exist in this field.

### SUMMARY

In one aspect, there is provided a chromogenic absorbent material for hemoglobin and glucose detection in an animal excretion, the chromogenic absorbent material comprising:
a benzidine-type compound;
an organic hydroperoxide, for catalyzing the oxidation of the benzidine-type compound in the presence of hemoglobin;
a first enzyme which is an oxidoreductase, for in situ generation of hydrogen peroxide in the presence of glucose;
a second enzyme which is a peroxidase, a pseudoperoxidase or a combination thereof, for catalyzing the oxidation of the benzidine-type compound upon in situ generation of the hydrogen peroxide; and
a polysaccharide matrix, comprising:
   from about 10 wt % to about 32.5 wt % chemically or mechanically processed non-functionalized cellulose;
   from about 25 wt % to about 70 wt % pregelatinized starch (PGS);
   from 0 wt % to about 20 wt % guar gum;
   from 0 wt % to about 25 wt % methyl hydroxyethyl cellulose (MHEC);
   from 0 wt % to about 15 wt % hydroxyethyl cellulose (HEC); and
   from 0 wt % to about 10 wt % carboxymethyl cellulose (CMC).

In another aspect, there is provided a chromogenic absorbent material for hemoglobin and glucose detection in an animal excretion, the chromogenic absorbent material comprising:
a benzidine-type compound;
at least one of:
   an organic hydroperoxide, for catalyzing the oxidation of the benzidine-type compound in the presence of hemoglobin; and
   a catalytic system comprising:
      a first enzyme which is an oxidoreductase, for in situ generation of hydrogen peroxide in the presence of glucose; and
      a second enzyme which is a peroxidase, a pseudoperoxidase or a combination thereof, for catalyzing the oxidation of the benzidine-type compound upon in situ generation of the hydrogen peroxide;
a salt of dodecylbenzene sulfonate; and
a polysaccharide matrix comprising a chemically or mechanically processed non-functionalized cellulose and pregelatinized starch (PGS).

### BRIEF DESCRIPTION OF THE FIGURES

FIG. 1 is a scheme of the reaction pathways taking place in a chromogenic absorbent material for the detection of glucose (left) and blood (right), according to one embodiment.

### DETAILED DESCRIPTION

The materials, methods and techniques described herein relate to a chromogenic absorbent material and the use for detecting blood (hemoglobin) and/or glucose in animal excretions.

Chromogenic absorbent material for detecting either blood or glucose in animal excretions were previously developed and are described, for example, in patent applications Pub. Nos. WO 2015/127528, WO 2016/049765 and WO 2017/165953, which are hereby incorporated by reference in their entirety. However, the chromogenic absorbent materials described in the above-mentioned patent applications only allowed detecting either blood or glucose depending on the chromogenic detection system incorporated within the polysaccharide matrix.

For blood (hemoglobin) detection, a polysaccharide matrix was impregnated with a chromogenic solution that included cumene hydroperoxide (CHP) and 3,3′,5,5′-tetramethylbenzidine (TMB) to form impregnated polysaccharide particles. The impregnated polysaccharide particles are then dried to form granules containing the CHP and the TMB. TMB turned blue in the presence of hemoglobin and CHP. For glucose detection, a polysaccharide matrix was impregnated with a chromogenic solution that included a catalytic system made of glucose oxidase (GOx) and horseradish peroxidase (HRP), and TMB to form impregnated polysaccharide particles. The impregnated polysaccharide particles are then dried to form granules containing GOx, HRP and TMB. The presence of glucose triggered in situ formation of hydrogen peroxide, which in turn turned TMB blue.

Manufacturing a chromogenic absorbent material able of detecting blood and glucose using a single chromogenic solution was initially thought to not be possible, due to the potential reactivity between cumene hydroperoxide and the catalytic system GOx/HRP. Indeed, early attempts at manufacturing such chromogenic absorbent material were unsuccessful, as the chromogenic solution itself or the chromogenic absorbent material would irreversibly turn blue, even in the absence of glucose or hemoglobin (false positive).

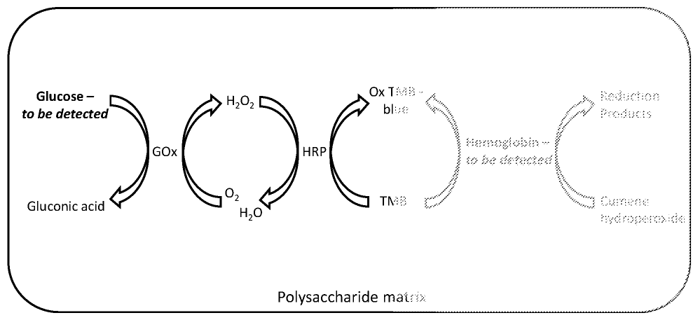

It was however surprisingly found that by selecting appropriate polysaccharide matrices, it was possible to avoid the false positive blue coloration and instead obtain a chromogenic absorbent material that was able of detecting blood and glucose using a single chromogenic solution. In some scenarios, the HRP and CHP contents can be tuned such that the components do not react together and generate a false positive. The resulting chromogenic material and the various components is schematically represented at FIG. 1.

In a first aspect of the description, various embodiments of such chromogenic absorbent materials are presented. Other aspects of the description further outline additional modifications made to the chromogenic absorbent materials.

The term “animal excretion”, as used herein, refers to urine or fecal matter excreted by an animal. The animal may be a cat, a dog, a rodent, a horse, a cow or any other livestock. The animal may alternatively be a human.

The term “chromogenic absorbent material”, as used herein, refers to an absorbent polysaccharide matrix able to absorb water, urine, aqueous solutions or organic solutions that include water (e.g., an acetone solution that includes some water) onto which a chromogenic solution is impregnated.

Particles of chromogenic absorbent material may, for example, be obtained as granules and can be used as standalone chromogenic absorbent material or in conjunction with an animal litter (e.g., the chromogenic absorbent material can be dispersed on a surface of the animal litter). The animal litter may include any suitable type of animal litter, such as clay-based litter, cellulosic litter, perlite-based litter, silica-based litter, corn-based litter, paper-based litter, wheat-based litter or any other organic-based litter, or a combination thereof. For example, the clay-based litter can be a bentonite litter and/or a montmorillonite litter.

In one aspect of the description, the chromogenic absorbent material for detection of hemoglobin and glucose in an animal excretion includes:
a chromogenic indicator;
a first oxidizing agent responsive to peroxidatic/pseudoperoxidatic activity in the animal excretion;
a catalytic system comprising:
   a first catalytic compound for in situ generation of a second oxidizing agent responsive to peroxidatic/pseudoperoxidatic activity in the animal excretion; and
   a second catalytic compound for catalyzing the oxidation of the chromogenic indicator upon in situ generation of the second oxidizing agent, wherein the chromogenic indicator is responsive to the first oxidizing agent and the second oxidizing agent; and
a polysaccharide matrix.

In another aspect of the description, the chromogenic absorbent material for detection of hemoglobin and glucose in an animal excretion includes:
a chromogenic indicator;
at least one of:
   a first oxidizing agent responsive to peroxidatic/pseudoperoxidatic activity in the animal excretion; and
   a catalytic system comprising:
      a first catalytic compound for in situ generation of a second oxidizing agent responsive to peroxidatic/pseudoperoxidatic activity in the animal excretion; and
      a second catalytic compound for catalyzing the oxidation of the chromogenic indicator upon in situ generation of the second oxidizing agent; and
a polysaccharide matrix,
wherein the chromogenic indicator is responsive to the first oxidizing agent and the second oxidizing agent.

The first oxidizing agent is responsive to peroxidatic/pseudoperoxidatic activity in the animal excretion, and the first catalytic compound generates a second oxidizing agent in situ. The second oxidizing agent is also responsive to peroxidatic/pseudoperoxidatic activity in the animal excretion.

It should be understood that the expression “peroxidatic activity” refers to the ability of catalytic compounds to drive the reaction between hydroperoxides and colorless chromogenic electron donors which become fluorescent or visibly colored after oxidation.

It should be understood that the expression “pseudoperoxidatic activity” refers to the ability of a peroxidase or a non-peroxidase catalytic compound to drive the reaction between hydroperoxidases and colorless chromogenic electron donors which become fluorescent or visibly colored after oxidation. Certain transition metals and their ions and hemoproteins are known to have pseudoperoxidatic activity. Basophils, neutrophils, eosinophils and mast cells synthesize endogenous peroxidase which can be visualized at the ultrastructural level in the secretory apparatus of immature cells. Red blood cells and hematin containing compounds have iron as part of their heme groups, which can catalyze the oxidation of chromogenic electron donors. This pseudoperoxidatic activity can be inhibited with strong H₂O₂ solutions, sodium azide and methanol-H₂O₂ solutions.

In some embodiments, the first oxidizing agent is an organic hydroperoxide of general formula ROOH, wherein the R group is an aryl, alkyl or acyl group. For example, and without being limitative, the organic hydroperoxide can be cumene hydroperoxide (CHP), diisopropylbenzene dihydroperoxide or a combination thereof. In some embodiments, the first oxidizing agent can be a hydroperoxide precursor such as sodium percarbonate. Sodium percarbonate is a chemical adduct of sodium carbonate and hydrogen peroxide, of formula 2Na₂CO₃·3H₂O₂. Sodium percarbonate decomposes to sodium carbonate and hydrogen peroxide, for example upon contact with an aqueous solution.

The second oxidizing agent is not initially added to the chromogenic absorbent material and is instead generated in situ by the first catalytic compound. It should be understood that the expression “generated in situ” means that the second oxidizing agent is directly synthesized in the chromogenic absorbent material from a precursor. For example, the first catalytic compound can be an enzyme such as an oxidoreductase. A non-limiting example of an oxidoreductase is glucose oxidase (GOx). The second oxidizing agent can, in this case, be hydrogen peroxide. A second catalytic compound is also present to enable the oxidation of the chromogenic indicator in the presence of glucose. A non-limiting example of a suitable second catalytic compound is horseradish peroxidase.

In some embodiments, the oxidizing activity of the first and/or second oxidizing agents is triggered by the presence of peroxidatic/pseudoperoxidatic activity in the animal excretions. The first and/or second oxidizing agents therefore oxidize the chromogenic indicator which then changes color. More particularly, the chromogenic indicator can be an electron donor, i.e., a reducing agent that changes color upon losing an electron.

In some embodiments, the chromogenic indicator is a benzidine-type compound, that is a compound as shown in Formula I:

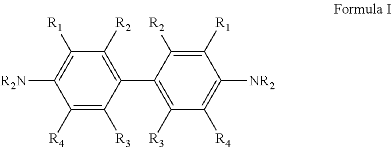

In Formula I, R₁, R₂, R₃ and R₄ may be the same or different and may be hydrogen, halogen, a lower alkyl or alkoxy group containing 1 to 4 carbon atoms, a (C₁-C₄)-dialkylamino group, an acetylamino group, a nitro group or an aromatic group which may be substituted.

Optionally, the chromogenic indicator may be a compound as shown in Formula II:

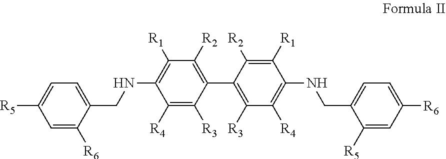

In Formula II, groups R₁, R₂, R₃ and R₄ may be the same or different and represent hydrogen, halogen, and a lower alkyl or alkoxy group containing 1 to 4 carbon atoms, a (C₁-C₄)-dialkylamino group, an acetylamino group, a nitro group or an aromatic group which may be substituted; R₅ and R₆ are the same or different and represent water-soluble groups as hydroxyl group, amino group, acidic group, disulfonyl group, ether group, halogen, and a lower alkyl or alkoxy group containing 1 to 4 carbon atoms, a (C₁-C₄)-dialkylamino group, an acetylamino group or a nitro group.

Thus, a water soluble benzidine-type chromogenic indicator of Formula II, responds in the presence of hydroperoxide and peroxidase by changing its light absorptive capability, which is due to the chemical transformation to the compound shown in Formula III:

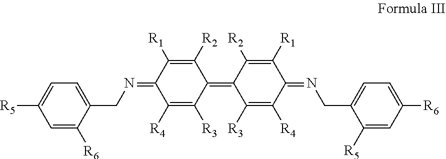

It is understood that different types of benzidine-type chromogenic indicators may be used.

Optionally, the benzidine-type compound may be 3,3′,5,5′-tetramethylbenzidine (TMB). TMB is a colorless agent, which turns blue upon oxidation. The peroxidase and/or pseudo-peroxidase catalysts can catalyze the oxidation of TMB by the first and/or the second oxidizing agent according to the following oxidation reaction.

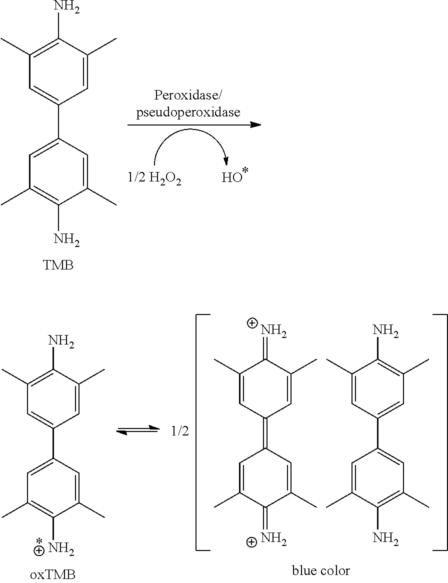

The polysaccharide matrix can be selected such that the first oxidizing agent and the catalytic system do not react with each other and do not yield a false positive. Most of the polysaccharide matrices described in the Examples of WO 2015/127528, WO 2016/049765 and WO 2017/165953 and used for glucose or hemoglobin detection included at least 35 wt % microcrystalline cellulose (MCC) and always yielded false positive results when contacted with a chromogenic solution that included both cumene hydroperoxide and a catalytic system including horseradish peroxidase (HRP) and glucose oxidase (GOx).

It was surprisingly found that when the polysaccharide matrix included less than about 35 wt % (preferably less than 32.5 wt %) of chemically or mechanically processed non-functionalized cellulose, no false positive result was obtained and detection of both glucose and hemoglobin was possible using a single granule including both an organic hydroperoxide and a catalytic system including horseradish peroxidase (HRP) and glucose oxidase (GOx) which was able to generate hydrogen peroxide in situ in the presence of glucose.

It was also surprisingly found that when the polysaccharide matrix included at least about 10 wt % methyl hydroxyethyl cellulose (MHEC), the surface of the granules featured a more homogeneous coloration when the chromogenic indicator was activated. This surprising effect was observed on chromogenic absorbent material able to detect hemoglobin alone, glucose alone or both hemoglobin and glucose.

It was also surprisingly found that when the polysaccharide matrix was free of anionic polymers (e.g., free of anionic polysaccharide and free of anionic superabsorbent polymer), no colored halo was observed on clumped litter particles neighbouring the chromogenic absorbent granules. This surprising effect was observed on chromogenic absorbent material able to detect hemoglobin alone, glucose alone or both hemoglobin and glucose.

In the description, it is understood that the expression “a chromogenic absorbent material for hemoglobin and glucose detection in an animal excretion” means that the chromogenic absorbent material is able to detect hemoglobin alone in an animal excretion, glucose alone in an animal excretion and hemoglobin and glucose simultaneously in an animal excretion. The chromogenic absorbent material for hemoglobin and glucose detection includes two chromogenic detection systems. The two chromogenic detection systems can make use of the same chromogenic indicator (e.g., a benzidine-type compound).

In the description, it is also understood that the expression “a chromogenic absorbent material for hemoglobin and/or glucose detection in an animal excretion” means that the chromogenic absorbent material is able to detect hemoglobin alone in an animal excretion, glucose alone in an animal excretion and/or hemoglobin and glucose simultaneously in an animal excretion. The chromogenic absorbent material for hemoglobin and/or glucose detection can include a single chromogenic system for glucose detection, a single chromogenic system for hemoglobin detection or two chromogenic systems for hemoglobin and glucose detection. When two chromogenic detection systems are used, the two chromogenic detection systems can make use of the same chromogenic indicator (e.g., a benzidine-type compound).

In some embodiments, the polysaccharide matrix includes from about 10 wt % to about 32.5 wt % chemically or mechanically processed non-functionalized cellulose, or from about 15 wt % to about 32.5 wt % chemically or mechanically processed non-functionalized cellulose, or from about 20 wt % to about 32.5 wt % chemically or mechanically processed non-functionalized cellulose, or from about 25 wt % to about 32.5 wt % chemically or mechanically processed non-functionalized cellulose, or from about 10 wt % to about 30 wt % chemically or mechanically processed non-functionalized cellulose, or from about 10 wt % to about 25 wt % chemically or mechanically processed non-functionalized cellulose, or from about 10 wt % to about 20 wt % chemically or mechanically processed non-functionalized cellulose, or from about 10 wt % to about 15 wt % chemically or mechanically processed non-functionalized cellulose. In some embodiments, the chemically or mechanically processed non-functionalized cellulose comprises microcrystalline cellulose (MCC), fibrous cellulose or a combination thereof.

In some embodiments, the polysaccharide matrix includes from about 10 wt % to about 32.5 wt % MCC, or from about 15 wt % to about 32.5 wt % MCC, or from about 20 wt % to about 32.5 wt % MCC, or from about 25 wt % to about 32.5 wt % MCC, or from about 10 wt % to about 30 wt % MCC, or from about 10 wt % to about 25 wt % MCC, or from about 10 wt % to about 20 wt % MCC, or from about 10 wt % to about 15 wt % MCC.

In some embodiments, the polysaccharide matrix includes from about 10 wt % to about 32.5 wt % fibrous cellulose, or from about 15 wt % to about 32.5 wt % fibrous cellulose, or from about 20 wt % to about 32.5 wt % fibrous cellulose, or from about 25 wt % to about 32.5 wt % MCC, or from about 10 wt % to about 30 wt % fibrous cellulose, or from about 10 wt % to about 25 wt % fibrous cellulose, or from about 10 wt % to about 20 wt % fibrous cellulose, or from about 10 wt % to about 15 wt % fibrous cellulose.

In some embodiments, the polysaccharide matrix includes from about 0 wt % to about 70 wt % pregelatinized starch (PGS), or from about 10 wt % to about 70 wt % PGS, or from about 20 wt % to about 70 wt % PGS, or from about 25 wt % to about 70 wt % PGS, or from about 30 wt % to about 70 wt % PGS, or from about 40 wt % to about 70 wt % PGS, or from about 50 wt % to about 70 wt % PGS, or from about 60 wt % to about 70 wt % PGS, or from about 65 wt % to about 70 wt % PGS, or from about 10 wt % to about 60 wt % PGS, or from about 10 wt % to about 50 wt % PGS, or from about 10 wt % to about 40 wt % PGS, or from about 10 wt % to about 30 wt % PGS, or from about 25 wt % to about 35 wt % PGS, or from about 55 wt % to about 60 wt % PGS, or from about 40 wt % to about 60 wt % PGS, or from about 45 wt % to about 55 wt % PGS.

In some embodiments, the polysaccharide matrix includes from 0 wt % to about 20 wt % guar gum, or from 0 wt % to about 15 wt % guar gum, or from 0 wt % to about 10 wt % guar gum, or from 0 wt % to about 5 wt % guar gum, or from about 5 wt % to about 20 wt % guar gum, or from about 10 wt % to about 20 wt % guar gum, or from about 15 wt % to about 20 wt % guar gum. In some embodiments, the polysaccharide matrix is free of guar gum.

In some embodiments, the polysaccharide matrix includes from 0 wt % to about 25 wt % methyl hydroxyethyl cellulose (MHEC), or from 0 wt % to about 20 wt % MHEC, or from 0 wt % to about 15 wt % MHEC, or from 0 wt % to about 10 wt % MHEC, or from 0 wt % to about 5 wt % MHEC, or from about 5 wt % to about 25 wt % MHEC, or from about 10 wt % to about 25 wt % MHEC, or from about 15 wt % to about 25 wt % MHEC, or from about 20 wt % to about 25 wt % MHEC, or from about 10 wt % to about 20 wt % MHEC, or from about 10 wt % to about 15 wt % MHEC, or from about 12.5 wt % to about 17.5 wt % MHEC, or from about 15 wt % to about 20 wt % MHEC. Preferably, the polysaccharide matrix includes at least about 10 wt % MHEC, or from about 10 wt % to about 20 wt % MHEC, or from about 12.5 wt % to about 17.5 wt % MHEC. In some embodiments, the MHEC is Tylose™, such as Tylose MH 60000 P6.

In some embodiments, the polysaccharide matrix includes from 0 wt % to about 15 wt % hydroxyethyl cellulose (HEC), or from 0 wt % to about 10 wt % HEC, or from 0 wt % to about 5 wt % HEC, or from about 5 wt % to about 15 wt % HEC, or from about 10 wt % to about 15 wt % HEC, or from about 5 wt % to about 10 wt % HEC.

In some embodiments, the polysaccharide matrix includes from 0 wt % to about 10 wt % carboxymethyl cellulose (CMC), or from 0 wt % to about 5 wt % CMC, or from about 2.5 wt % to about 7.5 wt % CMC. In some embodiments, the polysaccharide matrix is free of CMC.

In some embodiments, the polysaccharide matrix includes:
from about 10 wt % to about 32.5 wt % microcrystalline cellulose (MCC);
from about 25 wt % to about 70 wt % pregelatinized starch (PGS);
from 0 wt % to about 20 wt % guar gum;
from 0 wt % to about 25 wt % methyl hydroxyethyl cellulose (MHEC);
from 0 wt % to about 15 wt % hydroxyethyl cellulose (HEC); and
from 0 wt % to about 5 wt % carboxymethyl cellulose (CMC).

In some embodiments, the polysaccharide matrix includes:
from about 10 wt % to about 32.5 wt % MCC;
from about 25 wt % to about 70 wt % PGS;
from 0 wt % to about 20 wt % guar gum;
from 10 wt % to about 25 wt % MHEC; and
from 0 wt % to about 10 wt % HEC.

In some embodiments, the polysaccharide matrix consists essentially of:
from about 10 wt % to about 32.5 wt % MCC;
from about 25 wt % to about 70 wt % PGS;
from 0 wt % to about 20 wt % guar gum;
from 10 wt % to about 25 wt % MHEC; and
from 0 wt % to about 10 wt % HEC.

In some embodiments, the polysaccharide matrix consists essentially of:
from about 25 wt % to about 32.5 wt % MCC;
from about 25 wt % to about 35 wt % PGS;
from about 15 wt % to about 20 wt % guar gum; and
from about 15 wt % to about 25 wt % MHEC.

In some embodiments, the polysaccharide matrix consists essentially of:
from about 25 wt % to about 32.5 wt % MCC;
from about 55 wt % to about 60 wt % PGS; and
from about 10 wt % to about 15 wt % MHEC.

In some embodiments, the polysaccharide matrix consists essentially of:
from about 10 wt % to about 15 wt % MCC;
from about 65 wt % to about 70 wt % PGS;
from about 10 wt % to about 15 wt % MHEC; and
from about 5 wt % to about 10 wt % HEC.

In some embodiments, the polysaccharide matrix consists essentially of:
from about 25 wt % to about 32.5 wt % MCC;
from about 50 wt % to about 60 wt % PGS;
from about 10 wt % to about 15 wt % MHEC; and
from about 0 wt % to about 10 wt % HEC.

In some embodiments, the polysaccharide matrix includes:
a chemically or mechanically processed non-functionalized cellulose;
pregelatinized starch (PGS); and
at least 10 wt % methyl hydroxyethyl cellulose (MHEC), preferably from 10 wt % to about 20 wt % MHEC.

In some embodiments, the chemically or mechanically processed non-functionalized cellulose is microcrystalline cellulose or is a mechanically processed non-functionalized cellulose such as fibrous cellulose. In some embodiments, the microcrystalline cellulose has an average particle size by laser diffraction between about 25 μm and about 200 μm. In some embodiments, the mechanically processed non-functionalized cellulose can have an average particle size by laser diffraction between about 50 μm and about 80 μm, and a bulk density between about 0.10 g/mL and about 0.35 g/mL. In some embodiments, the mechanically processed non-functionalized cellulose can have an average fiber length between about 150 μm and about 250 μm, or between about 180 μm and about 220 μm, or of up to 220 μm. In some embodiments, the mechanically processed non-functionalized cellulose can have a bulk density between about 0.10 g/mL and about 0.35 g/mL, or between about 0.10 g/mL and about 0.30 g/mL, or between about 0.10 g/mL and about 0.25 g/mL, or between about 0.10 g/mL and about 0.20 g/mL, or between about 0.10 g/mL and about 0.15 g/mL, or between about 0.11 g/mL and about 0.145 g/mL.

In some embodiments, the chromogenic absorbent material includes an anionic surfactant, such as an alkylaryl sulfonate surfactant, an alkyl sulfate surfactant, an alkyl sulfonate surfactant, an aryl sulfonate surfactant or a combination thereof.

It was surprisingly found that when the chromogenic absorbent material included an alkylaryl sulfonate surfactant such as a salt of dodecylbenzene sulfonate (e.g., sodium dodecylbenzene sulfonate), the coloration of the chromogenic absorbent material was more stable and did not shift after several hours. This surprising effect was observed on chromogenic absorbent material able to detect hemoglobin alone, glucose alone or both hemoglobin and glucose.

In some embodiments, the alkylaryl sulfonate surfactant is a salt of an alkylbenzene sulfonate, such as a salt of dodecylbenzene sulfonate. Non-limiting examples of salts of dodecylbenzene sulfonate include sodium dodecylbenzene sulfonate, ammonium dodecylbenzene sulfonate, triethylamine dodecylbenzene sulfonate and potassium dodecylbenzene sulfonate.

In some embodiments, the chromogenic absorbent material may turn blue upon contact with excretions containing at least traces of blood (with therefore peroxidase/pseudo-peroxidase activity) and/or glucose. It should be understood that “blue” refers to any shade of blue. The chromogenic absorbent material may need a contact time with excretions sufficient to enable coloration. In an optional aspect, the particles may turn blue after a contact time ranging from about 10 seconds to about 30 min, or from about 10 seconds to about 1 min, depending on the nature of the polysaccharide matrix. In some embodiments, the chromogenic absorbent material may turn to different shades of blue depending on the blood and/or glucose concentration in excretions. The intensity of the blue shade may be proportional to the blood concentration or glucose concentration in the animal excretions.

In some embodiments, the chromogenic composition may further include a colour enhancer. Optionally, it may also include a buffering agent, a stabilizer, a metal scavenger agent or a combination thereof. The colour enhancer may optionally be a methoxyquinoline such as 6-methoxyquinoline, lepidine (4-methylquinoline), phenol derivatives, nitrobenzene, N-methylpyrrolidone, ethylene carbonate or any combination thereof. The buffering agent may optionally include citrate, sodium citrate, phosphate, acetate or any combination thereof. The stabilizer may optionally be dibutylhydroxytoluene (BHT), uric acid, ascorbic acid, ammonium molybdate, polyethylene glycol, polyvinylpyrrolidone, polyethylene oxide or derivatives thereof or a combination thereof. The metal-scavenger agent may optionally be EDTA, NTA, DTPA, STPP or a salt thereof, or any combination thereof.

In some embodiments, depending on the polysaccharide matrix, the chromogenic absorbent material may have a density of about 0.20 g/cm³ to about 0.39 g/cm³, of about 0.20 g/cm³ to about 0.35 g/cm³, of about 0.25 g/cm³ to about 0.35 g/cm³, or of about 0.30 g/cm³ to about 0.35 g/cm³.

In some embodiments, depending on the polysaccharide matrix, the chromogenic absorbent material may have a total porosity of about 65% to about 85%, or of about 70% to about 80%. It is understood that the total porosity refers to the fraction of the bulk material volume (V) which is not occupied by solid matter. If the volume of solids is denoted by Vs, and the pore volume as Vpore=V−Vs, the total porosity can be expressed as shown in Equation 1 below.

$$total porosity = ϕ = \frac{V - Vs}{V} = \frac{V pore}{V} ( mL / mL ) \qquad Equation 1$$

The total porosity may for example be measured by: placing a known volume of chromogenic absorbent particles into a container; covering the particles with a liquid; and measuring the volume of liquid needed to cover the particles (Vc). The total porosity is then expressed as the ratio of the volume of added liquid (Vc) to the volume of particles (V).

In some implementation, depending on the absorptive material, the particles of chromogenic absorbent material have an effective porosity of about 0.5 mL/g to about 2.0 mL/g, of about 0.6 mL/g to about 1.5 mL/g, of about 0.8 mL/g to about 1.2 mL/g or of about 0.9 mL/g to about 1.1 mL/g. It is understood that the effective porosity (also referred to as connected porosity or true porosity) is defined as the ratio of the connected pore volume to the total bulk volume. The effective porosity may for example be measured by: placing a known mass (m) of chromogenic absorbent particles into a container; covering the particles with a liquid; measuring the volume of liquid needed to cover the particles (Vc); removing the soaked particles from the container; measuring the liquid remaining in the container (Vr); and calculating the volume of liquid absorbed in the chromogenic absorbent particles (Va=Vc−Vr). The effective porosity may then be obtained as shown in Equation 2 below.

$$effective porosity = {ϕ}_{e} = \frac{Vc - Vr}{m} = \frac{Va}{m} ( ml / g ) \qquad Equation 2$$

It is to be noted that the effective porosity may also be expressed as the ratio Va/V in mL/mL.

In some implementations, the chromogenic absorbent material has a free swelling capacity (FSC) greater than about 900%, or greater than about 1000%. The FSC is one type of measurement used for measuring the absorption properties of a material. An FSC measurement is performed by soaking the material to be tested in a liquid to be absorbed (in the present case, water) for a given time and weighing the material after the liquid has been absorbed. In some implementations, the chromogenic absorbent material has a higher FSC than compared to the litter material. For example, the chromogenic absorbent material may have a FSC about 1.5 to 2 times higher than the FSC of the litter material.

Depending on the absorptive material, the particles of chromogenic absorbent material may have a pore density greater than about 20%, or greater than about 25%, or of about 27% to about 33%, for example. The pores of the particles of chromogenic absorbent material have an equivalent diameter greater than about 20 μm, or of about 20 μm to about 40 μm, or of about 20 μm to about 30 μm.

Particles of chromogenic absorbent material can be manufactured using the following process:
providing a polysaccharide matrix that is water-absorbing;
providing a chromogenic solution comprising a chromogenic indicator and at least one of a first oxidizing agent and a catalytic system comprising a first catalytic compound for in situ generation of a second oxidizing agent responsive to peroxidatic/pseudoperoxidatic activity in the animal excretion and a second catalytic compound for catalyzing the oxidation of the chromogenic indicator upon in situ generation of the second oxidizing agent;
impregnating the polysaccharide matrix with the chromogenic solution to obtain solution-impregnated humid particles; and
drying the solution-impregnated humid particles to obtain the chromogenic absorbent material.

The chromogenic solution may include additional components, as described herein.

In some embodiments, the chromogenic solution is poured (e.g., dripped in the form of discrete drops) onto the polysaccharide matrix to impregnate the polysaccharide matrix and form corresponding discrete solution-impregnated humid particles. Optionally, the solution-impregnated humid particles may be recovered by sieving the mixture of solution-impregnated humid particles and remaining polysaccharide matrix. The drying step may be performed under vacuum and/or at various temperatures ranging from about 15° C. to about 80° C. Using low-shear methods as described herein allows the particles of chromogenic absorbent material to be obtained as granules having a lower density, higher porosity compared with other types of particles obtained by methods such as extrusion or pressing. The granules are typically quasi-spherical and part of the surface of the granule can have a concave shape.

The description also provides the following embodiments:
1. A chromogenic absorbent material for hemoglobin and glucose detection in an animal excretion, the chromogenic absorbent material comprising:
   a first oxidizing agent responsive to peroxidatic/pseudoperoxidatic activity in the animal excretion, preferably, the first oxidizing agent is an organic hydroperoxide for catalyzing the oxidation of a benzidine-type compound in the presence of hemoglobin;
   a catalytic system comprising:
      a first catalytic compound for in situ generation of a second oxidizing agent responsive to peroxidatic/pseudoperoxidatic activity in the animal excretion, preferably, the first catalytic compound is an oxidoreductase, for in situ generation of hydrogen peroxide in the presence of glucose; and
      a second catalytic compound for catalyzing the oxidation of the chromogenic indicator upon in situ generation of the second oxidizing agent, preferably, the second catalytic compound is a peroxidase, a pseudoperoxidase or a mixture thereof, for catalyzing the oxidation of the benzidine-type compound upon in situ generation of the hydrogen peroxide;
   a chromogenic indicator responsive to the first oxidizing agent and the second oxidizing agent, preferably, the chromogenic indicator is a benzidine-type compound; and
   a polysaccharide matrix, comprising:
      from about 10 wt % to about 32.5 wt % chemically or mechanically processed non-functionalized cellulose, such as microcrystalline cellulose (MCC),
      fibrous cellulose or a combination thereof;
      from about 25 wt % to about 70 wt % pregelatinized starch (PGS);
      from 0 wt % to about 20 wt % guar gum;
      from 0 wt % to about 25 wt % methyl hydroxyethyl cellulose (MHEC);
      from 0 wt % to about 15 wt % hydroxyethyl cellulose (HEC); and
      from 0 wt % to about 10 wt %, or from 2.5 wt % to about 7.5 wt %, or from 0 wt % to about 5 wt % carboxymethyl cellulose (CMC).
2. The chromogenic absorbent material of embodiment 1, wherein the polysaccharide matrix comprises:
   from about 10 wt % to about 32.5 wt % MCC;
   from about 25 wt % to about 70 wt % PGS;
   from 0 wt % to about 20 wt % guar gum;
   from 10 wt % to about 25 wt % MHEC; and
   from 0 wt % to about 10 wt % HEC.
3. The chromogenic absorbent material of embodiment 2, wherein the polysaccharide matrix consists essentially of:
   from about 10 wt % to about 32.5 wt % MCC;
   from about 25 wt % to about 70 wt % PGS;
   from 0 wt % to about 20 wt % guar gum;
   from 10 wt % to about 25 wt % MHEC; and
   from 0 wt % to about 10 wt % HEC.
4. The chromogenic absorbent material of embodiment 3, wherein the polysaccharide matrix consists essentially of:
   from about 25 wt % to about 32.5 wt % MCC;
   from about 25 wt % to about 35 wt % PGS;
   from about 15 wt % to about 20 wt % guar gum; and
   from about 15 wt % to about 25 wt % MHEC.
5. The chromogenic absorbent material of embodiment 3, wherein the polysaccharide matrix consists essentially of:
   from about 25 wt % to about 32.5 wt % MCC;
   from about 55 wt % to about 60 wt % PGS; and
   from about 10 wt % to about 15 wt % MHEC.
6. The chromogenic absorbent material of embodiment 3, wherein the polysaccharide matrix consists essentially of:
   from about 10 wt % to about 15 wt % MCC;
   from about 65 wt % to about 70 wt % PGS;
   from about 10 wt % to about 15 wt % MHEC; and
   from about 5 wt % to about 10 wt % HEC.
7. The chromogenic absorbent material of embodiment 3, wherein the polysaccharide matrix consists essentially of:
   from about 25 wt % to about 32.5 wt % MCC;
   from about 50 wt % to about 60 wt % PGS;
   from about 10 wt % to about 15 wt % MHEC; and
   from about 0 wt % to about 10 wt % HEC.
8. The chromogenic absorbent material of embodiment 1, wherein the polysaccharide matrix comprises:
   from about 10 wt % to about 32.5 wt % chemically or mechanically processed non-functionalized cellulose;
   from about 25 wt % to about 70 wt % PGS;
   from about 10 wt % to about 20 wt % MHEC; and
   from about 2.5 wt % to about 7.5 wt % CMC.
9. The chromogenic absorbent material of embodiment 8, wherein the polysaccharide matrix comprises:
   from about 25 wt % to about 32.5 wt % chemically or mechanically processed non-functionalized cellulose;
   from about 40 wt % to about 60 wt % PGS;
   from about 10 wt % to about 20 wt % MHEC; and
   from about 2.5 wt % to about 7.5 wt % CMC.
10. The chromogenic absorbent material of embodiment 9, wherein the polysaccharide matrix consists essentially of:
   from about 25 wt % to about 32.5 wt % chemically or mechanically processed non-functionalized cellulose;
   from about 40 wt % to about 60 wt % PGS;
   from about 10 wt % to about 20 wt % MHEC; and
   from about 2.5 wt % to about 7.5 wt % CMC.
11. The chromogenic absorbent material of embodiment 9, wherein the polysaccharide matrix consists essentially of:
   from about 25 wt % to about 32.5 wt % chemically or mechanically processed non-functionalized cellulose;
   from about 45 wt % to about 55 wt % PGS;
   from about 12.5 wt % to about 17.5 wt % MHEC; and
   from about 2.5 wt % to about 7.5 wt % CMC.
12. The chromogenic absorbent material of any one of embodiments 1 to 11, wherein the chemically or mechanically processed non-functionalized cellulose comprises microcrystalline cellulose (MCC), fibrous cellulose or a combination thereof.
13. The chromogenic absorbent material of embodiment 12, wherein the microcrystalline cellulose has an average particle size by laser diffraction between about 25 μm and about 200 μm, and wherein the fibrous cellulose has an average fiber length of up to about 220 μm.
14. The chromogenic absorbent material of embodiment 12, wherein the chemically or mechanically processed non-functionalized cellulose is a mechanically processed non-functionalized cellulose having an average particle size by laser diffraction between about 50 μm and about 80 μm, and a bulk density between about 0.20 g/mL and about 0.35 g/mL.
15. The chromogenic absorbent material of any one of embodiments 1 to 14, further comprising an anionic surfactant.
16. The chromogenic absorbent material of embodiment 15, wherein the anionic surfactant is an alkylaryl sulfonate surfactant.
17. The chromogenic absorbent material of embodiment 16, wherein the alkylaryl sulfonate surfactant is a salt of an alkylbenzene sulfonate.
18. The chromogenic absorbent material of embodiment 17, wherein the salt of the alkylbenzene sulfonate is a salt of dodecylbenzene sulfonate.
19. The chromogenic absorbent material of embodiment 18, wherein the salt of dodecylbenzene sulfonate is sodium dodecylbenzene sulfonate, ammonium dodecylbenzene sulfonate, triethylamine dodecylbenzene sulfonate, potassium dodecylbenzene sulfonate or a combination thereof.
20. A chromogenic absorbent material for hemoglobin and/or glucose detection in an animal excretion, the chromogenic absorbent material comprising:
   at least one of:
      a first oxidizing agent responsive to peroxidatic/pseudoperoxidatic activity in the animal excretion, preferably, the first oxidizing agent is an organic hydroperoxide for catalyzing the oxidation of a benzidine-type compound in the presence of hemoglobin; and
      a catalytic system comprising:
         a first catalytic compound for in situ generation of a second oxidizing agent responsive to peroxidatic/pseudoperoxidatic activity in the animal excretion, preferably, the first catalytic compound is an oxidoreductase, for in situ generation of hydrogen peroxide in the presence of glucose; and
         a second catalytic compound for catalyzing the oxidation of the chromogenic indicator upon in situ generation of the second oxidizing agent, preferably, the second catalytic compound is a peroxidase, a pseudoperoxidase or a mixture thereof, for catalyzing the oxidation of the benzidine-type compound upon in situ generation of the hydrogen peroxide;
   a chromogenic indicator responsive to the first oxidizing agent and the second oxidizing agent, preferably, the chromogenic indicator is a benzidine-type compound;
   a salt of dodecylbenzene sulfonate; and
   a polysaccharide matrix comprising a chemically or mechanically processed non-functionalized cellulose and pregelatinized starch (PGS).
21. The chromogenic absorbent material of embodiment 20, wherein the polysaccharide matrix comprises:
   from about 10 wt % to about 32.5 wt % chemically or mechanically processed non-functionalized cellulose;
   from about 25 wt % to about 70 wt % PGS;
   from 0 wt % to about 20 wt % guar gum;
   from 0 wt % to about 25 wt % methyl hydroxyethyl cellulose (MHEC);
   from 0 wt % to about 15 wt % hydroxyethyl cellulose (HEC); and
   from 0 wt % to about 10 wt % carboxymethyl cellulose (CMC).
22. The chromogenic absorbent material of embodiment 21, wherein the polysaccharide matrix comprises:
   from about 10 wt % to about 32.5 wt % chemically or mechanically processed non-functionalized cellulose;
   from about 25 wt % to about 70 wt % PGS;
   from about 10 wt % to about 20 wt % MHEC; and
   from about 2.5 wt % to about 7.5 wt % CMC.
23. The chromogenic absorbent material of embodiment 21, wherein the polysaccharide matrix comprises:
   from about 25 wt % to about 32.5 wt % chemically or mechanically processed non-functionalized cellulose;
   from about 40 wt % to about 60 wt % PGS;
   from about 10 wt % to about 20 wt % MHEC; and
   from about 2.5 wt % to about 7.5 wt % CMC.
24. The chromogenic absorbent material of embodiment 23, wherein the polysaccharide matrix consists essentially of:
   from about 25 wt % to about 32.5 wt % chemically or mechanically processed non-functionalized cellulose;
   from about 40 wt % to about 60 wt % PGS;
   from about 10 wt % to about 20 wt % MHEC; and
   from about 2.5 wt % to about 7.5 wt % CMC.
25. The chromogenic absorbent material of embodiment 23, wherein the polysaccharide matrix consists essentially of:
   from about 25 wt % to about 32.5 wt % chemically or mechanically processed non-functionalized cellulose;
   from about 45 wt % to about 55 wt % PGS;
   from about 12.5 wt % to about 17.5 wt % MHEC; and
   from about 2.5 wt % to about 7.5 wt % CMC.
26. The chromogenic absorbent material of any one of embodiments 21 to 25, wherein the chemically or mechanically processed non-fonctionalized cellulose is microcrystalline cellulose (MCC), fibrous cellulose, or a mixture thereof.
27. The chromogenic absorbent material of embodiment 26, wherein the microcrystalline cellulose has an average particle size by laser diffraction between about 25 μm and about 200 μm, and wherein the fibrous cellulose has an average fiber length of up to about 220 μm.
28. The chromogenic absorbent material of any one of embodiments 21 to 25, wherein the chemically or mechanically processed non-fonctionalized cellulose is a mechanically processed non-functionalized cellulose having an average particle size by laser diffraction between about 50 μm and about 80 μm, and a bulk density between about 0.20 g/mL and about 0.35 g/mL.
29. The chromogenic absorbent material of any one of embodiments 21 to 28, wherein the salt of dodecylbenzene sulfonate is sodium dodecylbenzene sulfonate, ammonium dodecylbenzene sulfonate, triethylamine dodecylbenzene sulfonate, potassium dodecylbenzene sulfonate or a mixture thereof.
30. The chromogenic absorbent material of any one of embodiments 1 to 29, wherein the first catalytic compound is glucose oxidase (GOx) and the second catalytic compound is horseradish peroxidase (HRP).
31. The chromogenic absorbent material of any one of embodiments 1 to 30, wherein the benzidine-type compound comprises 3,3′,5,5′-tetramethylbenzidine.
32. The chromogenic absorbent material of any one of embodiments 1 to 31, wherein the organic hydroperoxide is cumene hydroperoxide, diisopropylbenzene dihydroperoxide, or a combination thereof.
33. The chromogenic absorbent material of any one of embodiments 1 to 32, further comprising a buffering agent, a stabilizer, a metal scavenger, a color enhancer or a combination thereof.
34. The chromogenic absorbent material of embodiment 33, wherein the color enhancer comprises 6-methoxyquinoline, lepidine, phenol derivatives, nitrobenzene, N-methylpyrrolidone or ethylene carbonate or a combination thereof.
35. The chromogenic absorbent material of embodiment 33 or 34, wherein the buffering agent comprises citrate, sodium citrate, phosphate or acetate or a combination thereof.
36. The chromogenic absorbent material of any one of embodiments 33 to 35, wherein the stabilizer comprises dibutylhydroxytoluene (BHT), uric acid, ascorbic acid, ammonium molybdate, polyethylene glycol, polyvinylpyrrolidone, polyethylene oxide or derivatives thereof or a combination thereof.
37. The chromogenic absorbent material of any one of embodiments 33 to 36, wherein the metal scavenger agent comprises ethylenediaminetetraacetic acid (EDTA) or EDTA sodium salt or a combination thereof.
38. The chromogenic absorbent material of any one of embodiments 1 to 37, having a density of about 0.20 g/cm³ to about 0.39 g/cm³.
39. The chromogenic absorbent material of any one of embodiments 1 to 38, material having an effective porosity of about 0.5 mL/g to about 2.0 mL/g.
40. The chromogenic absorbent material of any one of embodiments 1 to 39, which has pores having an equivalent diameter greater than about 20 μm.
41. The chromogenic absorbent material of any one of embodiments 1 to 40, having a free swelling capacity greater than about 900%.
42. The chromogenic absorbent material of any one of embodiments 1 to 41, having a pore density greater than about 20%.
43. A chromogenic absorbent material for hemoglobin and/or glucose detection in an animal excretion, the chromogenic absorbent material comprising:
   at least one of:
      a first oxidizing agent responsive to peroxidatic/pseudoperoxidatic activity in the animal excretion; and
      a catalytic system comprising:
         a first catalytic compound for in situ generation of a second oxidizing agent responsive to peroxidatic/pseudoperoxidatic activity in the animal excretion; and
         a second catalytic compound for catalyzing the oxidation of the chromogenic indicator upon in situ generation of the second oxidizing agent;
   a chromogenic indicator responsive to the first oxidizing agent and the second oxidizing agent; and
   a polysaccharide matrix, comprising:
      a chemically or mechanically processed non-functionalized cellulose;
      pregelatinized starch (PGS); and
      at least 10 wt % methyl hydroxyethyl cellulose (MHEC).
44. The chromogenic absorbent material of embodiment 43, wherein the polysaccharide matrix further comprises hydroxyethyl cellulose (HEC).
45. The chromogenic absorbent material of embodiment 43 or 44, wherein the chemically or mechanically processed non-fonctionalized cellulose is microcrystalline cellulose.
46. The chromogenic absorbent material of embodiment 43 or 44, wherein the chemically or mechanically processed non-fonctionalized cellulose is a mechanically processed non-functionalized cellulose having an average particle size by laser diffraction between about 50 μm and about 80 μm, and a bulk density between about 0.20 g/mL and about 0.35 g/mL.
47. The chromogenic absorbent material of any one of embodiments 43 to 46, wherein the polysaccharide matrix comprises between 10 wt % and about 20 wt % MHEC.
48. The chromogenic absorbent material of any one of embodiments 43 to 47, further comprising an alkylaryl sulfonate surfactant.
49. The chromogenic absorbent material of embodiment 48, wherein the alkylaryl sulfonate surfactant is a salt of an alkylbenzene sulfonate.
50. The chromogenic absorbent material of embodiment 48, wherein the salt of the alkylbenzene sulfonate is a salt of dodecylbenzene sulfonate.
51. The chromogenic absorbent material of embodiment 50, wherein the salt of dodecylbenzene sulfonate is sodium dodecylbenzene sulfonate, ammonium dodecylbenzene sulfonate, triethylamine dodecylbenzene sulfonate or potassium dodecylbenzene sulfonate.
52. A chromogenic absorbent material for hemoglobin and/or glucose detection in an animal excretion, the chromogenic absorbent material comprising:
   at least one of:
      a first oxidizing agent responsive to peroxidatic/pseudoperoxidatic activity in the animal excretion; and
      a catalytic system comprising:
         a first catalytic compound for in situ generation of a second oxidizing agent responsive to peroxidatic/pseudoperoxidatic activity in the animal excretion; and
         a second catalytic compound for catalyzing the oxidation of the chromogenic indicator upon in situ generation of the second oxidizing agent;
   a chromogenic indicator responsive to the first oxidizing agent and the second oxidizing agent;
   an alkylaryl sulfonate surfactant; and
   a polysaccharide matrix.
53. The chromogenic absorbent material of embodiment 52, wherein the polysaccharide matrix comprises:
   from about 10 wt % to about 32.5 wt % microcrystalline cellulose (MCC);
   from about 25 wt % to about 70 wt % pregelatinized starch (PGS);
   from 0 wt % to about 20 wt % guar gum;
   from 0 wt % to about 25 wt % methyl hydroxyethyl cellulose (MHEC);
   from 0 wt % to about 15 wt % hydroxyethyl cellulose (HEC); and
   from 0 wt % to about 5 wt % carboxymethyl cellulose (CMC).
54. The chromogenic absorbent material of embodiment 53, wherein the polysaccharide matrix comprises:
   from about 10 wt % to about 32.5 wt % MCC;
   from about 25 wt % to about 70 wt % PGS;
   from 0 wt % to about 20 wt % guar gum;
   from 10 wt % to about 25 wt % MHEC; and
   from 0 wt % to about 10 wt % HEC.
55. The chromogenic absorbent material of embodiment 54, wherein the polysaccharide matrix consists essentially of:
   from about 10 wt % to about 32.5 wt % MCC;
   from about 25 wt % to about 70 wt % PGS;
   from 0 wt % to about 20 wt % guar gum;
   from 10 wt % to about 25 wt % MHEC; and
   from 0 wt % to about 10 wt % HEC.
56. The chromogenic absorbent material of embodiment 55, wherein the polysaccharide matrix consists essentially of:
   from about 25 wt % to about 32.5 wt % MCC;
   from about 25 wt % to about 35 wt % PGS;
   from about 15 wt % to about 20 wt % guar gum; and
   from about 15 wt % to about 25 wt % MHEC.
57. The chromogenic absorbent material of embodiment 55, wherein the polysaccharide matrix consists essentially of:
   from about 25 wt % to about 32.5 wt % MCC;
   from about 55 wt % to about 60 wt % PGS; and
   from about 10 wt % to about 15 wt % MHEC.
58. The chromogenic absorbent material of embodiment 55, wherein the polysaccharide matrix consists essentially of:
   from about 10 wt % to about 15 wt % MCC;
   from about 65 wt % to about 70 wt % PGS;
   from about 10 wt % to about 15 wt % MHEC; and
   from about 5 wt % to about 10 wt % HEC.
59. The chromogenic absorbent material of embodiment 55, wherein the polysaccharide matrix consists essentially of:
   from about 25 wt % to about 32.5 wt % MCC;
   from about 50 wt % to about 60 wt % PGS;
   from about 10 wt % to about 15 wt % MHEC; and
   from about 0 wt % to about 10 wt % HEC.
60. The chromogenic absorbent material of embodiment 52, wherein the polysaccharide matrix comprises:
   a chemically or mechanically processed non-functionalized cellulose;
   pregelatinized starch (PGS); and
   at least 10 wt % methyl hydroxyethyl cellulose (MHEC).
61. The chromogenic absorbent material of embodiment 60, wherein the polysaccharide matrix further comprises hydroxyethyl cellulose (HEC).
62. The chromogenic absorbent material of embodiment 60 or 61, wherein the chemically or mechanically processed non-fonctionalized cellulose is microcrystalline cellulose.
63. The chromogenic absorbent material of embodiment 60 or 61, wherein the chemically or mechanically processed non-fonctionalized cellulose is a mechanically processed non-functionalized cellulose having an average particle size by laser diffraction between about 50 μm and about 80 μm, and a bulk density between about 0.20 g/mL and about 0.35 g/mL.
64. The chromogenic absorbent material of any one of embodiments 60 to 63, wherein the polysaccharide matrix comprises between 10 wt % and about 20 wt % MHEC.
65. The chromogenic absorbent material of any one of embodiments 52 to 64, wherein the alkylaryl sulfonate surfactant is a salt of an alkylbenzene sulfonate.
66. The chromogenic absorbent material of embodiment 65, wherein the salt of the alkylbenzene sulfonate is a salt of dodecylbenzene sulfonate.
67. The chromogenic absorbent material of embodiment 66, wherein the salt of dodecylbenzene sulfonate is sodium dodecylbenzene sulfonate, ammonium dodecylbenzene sulfonate, triethylamine dodecylbenzene sulfonate or potassium dodecylbenzene sulfonate.
68. A chromogenic absorbent material for hemoglobin and/or glucose detection in an animal excretion, the chromogenic absorbent material comprising:
   at least one of:
      a first oxidizing agent responsive to peroxidatic/pseudoperoxidatic activity in the animal excretion; and
      a catalytic system comprising:
         a first catalytic compound for in situ generation of a second oxidizing agent responsive to peroxidatic/pseudoperoxidatic activity in the animal excretion; and
         a second catalytic compound for catalyzing the oxidation of the chromogenic indicator upon in situ generation of the second oxidizing agent;
   a chromogenic indicator responsive to the first oxidizing agent and the second oxidizing agent; and
   a polysaccharide matrix which is free of anionic polymers.
69. The chromogenic absorbent material of embodiment 68, wherein the alkylaryl sulfonate surfactant is a salt of an alkylbenzene sulfonate.
70. The chromogenic absorbent material of embodiment 69, wherein the salt of the alkylbenzene sulfonate is a salt of dodecylbenzene sulfonate.
71. The chromogenic absorbent material of embodiment 70, wherein the salt of dodecylbenzene sulfonate is sodium dodecylbenzene sulfonate, ammonium dodecylbenzene sulfonate, triethylamine dodecylbenzene sulfonate or potassium dodecylbenzene sulfonate.
72. The chromogenic absorbent material of any one of embodiments 43 to 72, wherein the first oxidizing agent comprises an organic hydroperoxide.
73. The chromogenic absorbent material of embodiment 72, wherein the organic hydroperoxide is cumene hydroperoxide or diisopropylbenzene dihydroperoxide, or a combination thereof.
74. The chromogenic absorbent material of any one of embodiments 43 to 73, wherein: the first catalytic compound comprises an oxidoreductase enzyme;
   the second catalytic compound comprises a peroxidase, a pseudoperoxidase, or a combination thereof; and
   the second oxidizing agent is hydrogen peroxide.
75. The chromogenic absorbent material of embodiment 74, wherein the first catalytic compound is glucose oxidase (GOx) and the second catalytic compound is horseradish peroxidase (HRP).
76. The chromogenic absorbent material of any one of embodiments 43 to 75, wherein the chromogenic indicator comprises a benzidine-type compound.
77. The chromogenic absorbent material of embodiment 76, wherein the benzidine-type compound comprises 3,3′,5,5′-tetramethylbenzidine.
78. The chromogenic absorbent material of any one of embodiments 43 to 77, further comprising a buffering agent, a stabilizer, a metal scavenger, a color enhancer or a combination thereof.
79. The chromogenic absorbent material of embodiment 78, wherein the color enhancer comprises 6-methoxyquinoline, lepidine, phenol derivatives, nitrobenzene, N-methylpyrrolidone or ethylene carbonate or a combination thereof.
80. The chromogenic absorbent material of embodiment 78 or 79, wherein the buffering agent comprises citrate, sodium citrate, phosphate or acetate or a combination thereof.
81. The chromogenic absorbent material of any one of embodiments 78 to 80, wherein the stabilizer comprises dibutylhydroxytoluene (BHT), uric acid, ascorbic acid, ammonium molybdate, polyethylene glycol, polyvinylpyrrolidone, polyethylene oxide or derivatives thereof or a combination thereof.
82. The chromogenic absorbent material of any one of embodiments 78 to 81, wherein the metal scavenger agent comprises ethylenediaminetetraacetic acid (EDTA) or EDTA sodium salt or a combination thereof.
83. The chromogenic absorbent material of any one of embodiments 43 to 82, having a density of about 0.20 g/cm³ to about 0.39 g/cm³.
84. The chromogenic absorbent material of any one of embodiments 43 to 82, material having an effective porosity of about 0.5 mL/g to about 2.0 mL/g.
85. The chromogenic absorbent material of any one of embodiments 43 to 84, which has pores having an equivalent diameter greater than about 20 μm.
86. The chromogenic absorbent material of any one of embodiments 43 to 85, having a free swelling capacity greater than about 900%.
87. The chromogenic absorbent material of any one of embodiments 43 to 86, having a pore density greater than about 20%.

### EXAMPLES

### Urine Samples

Cat urine utilized in this evaluation was generously donated by Proanima (Boucherville). Urinary characteristics, notably leucocytes, nitrites, proteins, ketones, urobilinogen and bilirubin were evaluated using Roche Chemstrips® 9. The pH and USG were accurately determined using a pH-meter and refractometer, respectively.

Typical healthy feline urine was used for this evaluation, devoid of glucose and blood, with a pH of 6.3 and a USG of 1.030 g/mL.

### Glucose Solutions

A D-glucose stock solution was prepared in healthy feline urine at 1000 mg/dL. Subsequent glucose solutions were prepared by spiking healthy feline urine with the glucose stock at final concentrations of 0, 25, 50, 100, 150 and 300 mg/dL.

### Hemoglobin Solutions

A concentrated aqueous bovine hemoglobin (Hb) stock was first prepared in demineralized water. A secondary stock was prepared by diluting this aqueous stock in healthy feline urine. Subsequent hemoglobin solutions were prepared by spiking healthy feline urine with the Hb stock in urine, at final concentrations of 0, 30, 60, 90, 150 and 300 RBC/mL (RBC=red blood cell). Addition of aqueous Hb in the hemoglobin solutions did not constitute more than 2% of the total volume so as to not alter the inherent composition of the biological matrix.

### Granule Performance Assessment

Hemoglobin detection assessment: triplicate sets of granules were placed on a mineral-based litter, for each hemoglobin concentration. Three drops (˜ 150 μL) of hemoglobin solution were applied on each granule for each respective RBC concentration level. Dry control granules were also examined under identical conditions to ensure that no spontaneous color change (false positive) would occur throughout the duration of the assessment.

Glucose detection assessment: Triplicate sets of granules were placed on a mineral-based litter, for each glucose concentration. Three drops (˜ 150 μL) of sample were applied on each granule for each respective glucose concentration level. Dry control granules were also examined under identical conditions to ensure that no spontaneous color change (false positive) would occur throughout the duration of the assessment.

Digital images were captured at specified timepoints, notably at 1, 3, 5, 10, 20, 30 minutes well as at 1, 2 and 24 hours. All images in this study were captured in a LED light box (Photo Studio, Model 2x15-200-16-05, Output: 2×0.3 A) using a Sony digital camera (Mod ILCE-6000L). Acquisition parameters and settings were identical for all pictures and consisted of a 37 mm zoom, ISO of 100, 1/13 s. shutter speed and a focal aperture of 20 mm.

### Example 1 (Comparative)

Experiments were conducted to manufacture and assess granules made of previous generation polysaccharide matrices, as described in patent applications publication Nos. WO 2017/165953, WO 2016/049765 and WO 2015/127528, and by combining previous generation chromogenic solutions for hemoglobin and glucose detection.

The composition of the trial chromogenic solution is shown at Table 1A:

TABLE 1A
Trial chromogenic solution 1A for
glucose and hemoglobin detection

| Compound | wt % |
|---|---|
| Acetone | 44.03 |
| Acetate buffer pH 5.6 | 54.50 |
| Tetramethylbenzidine (TMB) | 0.44 |
| Butylated hydroxytoluene (BHT) | 0.01 |
| 6-Methoxyquinoline | 0.19 |
| Lepidine | 0.26 |
| Cumene hydroperoxide (CHP) | 0.47 |
| EDTA | 0.07 |
| Glucose oxidase | about 0.02 |
| Horseradish peroxidase | about 0.01 |

The composition of the polysaccharide matrices tested are shown at Table 1B:

TABLE 1B
Polysaccharide matrices 1a and 1b

| | Polysaccharide | Polysaccharide |
|---|---|---|
| Compound | Matrix 1a (wt %) | Matrix 1b (wt %) |
| Microcrystalline cellulose (MCC) | 49 | 60 |
| Pregelatinized Starch (PGS) | 49 | 5 |
| Sodium polyacrylate | 1 | 0 |
| superabsorbent polymer | | |
| Guar Gum | 0 | 10 |
| Carboxymethyl cellulose (CMC) | 0 | 25 |

For both polysaccharide matrices 1a and 1b: chromogenic granules were produced by dropwise addition of chromogenic solution 1A directly onto a powder bed of homogeneously prepared polysaccharide matrix 1a or 1b. This resulted in humid, quasi-spherical granules. The humid granules were immediately transferred onto a strainer to sift out any excess powder matrix and subsequently placed in an oven for drying at 70° C. The granules thereby obtained were then tested for glucose detection using a glucose solution and for hemoglobin detection using a hemoglobin solution.

For granules formed of polysaccharide matrix 1a: blue coloration of increasing intensity was observed with increasing hemoglobin concentrations between 30 and 300 RBC/μL; blue coloration of increasing intensity was observed with increasing glucose concentrations between 25 and 300 mg/dL, However, a false positive result was observed when demineralized water was poured onto the granules (hemoglobin concentration=0 RBC/μL; glucose concentration=0 mg/dL); and

For granules formed of polysaccharide matrix 1b: blue coloration of increasing intensity was observed with increasing hemoglobin concentrations between 30 and 300 RBC/μL; blue coloration of increasing intensity was observed with increasing glucose concentrations between 25 and 300 mg/dL, However, a false positive result was observed when demineralized water was poured onto the granules (hemoglobin concentration=0 RBC/μL; glucose concentration=0 mg/dL).

None of polysaccharide matrices 1a and 1b, when combined with chromogenic solution 1A, allowed obtaining granules that can be used for detecting both hemoglobin and glucose in cat urine.

### Example 2

Experiments were conducted to manufacture and assess granules including less than 35 wt % microcrystalline cellulose, using the chromogenic solution shown at Table 2A:

TABLE 2A
Chromogenic solution 2A for glucose and hemoglobin detection

| Compound | (g) |
|---|---|
| Water | 30.8 |
| Acetone | 19.757 |
| Tetramethylbenzidine (TMB) | 0.09 |
| Butylated hydroxytoluene (BHT) | 0.055 |
| 6-Methoxyquinoline | 0.0935 |
| Lepidine | 0.1325 |
| Cumene hydroperoxide (CHP) | 0.3060 |
| 10% w/w sodium dodecylbenzene | 500 μL |
| sulfonate (SDBS) | |
| EDTA | 0.0335 |
| Glucose oxidase | about 0.0016 |
| Horseradish peroxidase | about 0.0010 |

The composition of the polysaccharide matrices tested are shown at Table 2B:

TABLE 2B
Polysaccharide matrices 2a and 2b

| | Polysaccharide | Polysaccharide |
|---|---|---|
| Compound | Matrix 2a (wt %) | Matrix 2b (wt %) |
| Microcrystalline cellulose (MCC) | 32.5 | 25 |
| Pregelatinized Starch (PGS) | 50 | 45 |
| Hydroxyethyl cellulose | 0 | 5 |
| Guar Gum | 15 | 20 |
| Carboxymethyl cellulose (CMC) | 2.5 | 5 |

For both polysaccharide matrices 2a and 2b: chromogenic granules were produced by dropwise addition of chromogenic solution 2A directly onto a powder bed of homogeneously prepared polysaccharide matrix 2a or 2b. This resulted in humid, quasi-spherical granules 2a and 2b. The humid granules were immediately transferred onto a strainer to sift out any excess powder matrix and subsequently placed in an oven for drying at 70° C. The granules thereby obtained were then tested for glucose detection using a glucose solution and for hemoglobin detection using a hemoglobin solution.

Hemoglobin Detection with Granules 2a

Granules 2a rapidly displayed a blue coloration, seconds after application of the hemoglobin solutions at concentrations between 60 and 300 RBC/μL. Coloration lasted about 1 hour when SDBS was not present in the chromogenic solution and lasted for at least 24 hours when SDBS was present in the chromogenic solution, as shown in Table 2A. Contrary to the granules obtained in Example 1, no false positive coloration was observed when demineralized water was poured onto granules 2a.

Hemoglobin Detection with Granules 2b

Granules 2b performed similarly to granules 2a and rapidly displayed a blue coloration, seconds after application of the hemoglobin solutions at concentrations between 90 and 300 RBC/μL. Coloration lasted about 1 hour when SDBS was not present in the chromogenic solution and lasted for at least 24 hours when SDBS was present in the chromogenic solution, as shown in Table 2A. Contrary to the granules obtained in Example 1, no false positive coloration was observed when demineralized water was poured onto granules 2b.

Glucose Detection with Granules 2a

Granules 2a rapidly displayed a blue coloration, seconds after application of glucose solutions at concentrations as low as 25 mg/dL and also exhibited semi-quantitative characteristics. Coloration lasted about 1-2 hours when SDBS was not present in the chromogenic solution and lasted for at least 24 hours when SDBS was present in the chromogenic solution, as shown in Table 2A. Contrary to the granules obtained in Example 1, no false positive coloration was observed when demineralized water was poured onto granules 2a. However, only one side of granules 2a exhibited the blue coloration (i.e., the side that was first contacted with the chromogenic solution during formation of the granules). This phenomenon will be referred to herein as the candy effect. Without being bound by theory, the candy effect suggested that the glucose oxidase and horseradish peroxidase did not penetrate the entirety of the granules. While granules exhibiting the candy effect are functional,—in that a blue coloration is still visible—it is preferable that the candy effect be minimized or eliminated.

Glucose Detection with Granules 2b

Granules 2b performed similarly to granules 2a and rapidly displayed a blue coloration, seconds after application of glucose solutions at concentrations as low as 50 mg/dL. Coloration lasted about 1-2 hours when SDBS was not present in the chromogenic solution and lasted for at least 24 hours when SDBS was present in the chromogenic solution, as shown in Table 2A. Contrary to the granules obtained in Example 1, no false positive coloration was observed when demineralized water was poured onto granules 2b. Granules 2b also exhibited the candy effect.

### Example 3

Experiments were conducted to manufacture and assess additional granule formulations that included methyl hydroxyethyl cellulose, using the chromogenic solution shown at Table 3A.

TABLE 3A
Chromogenic solution 3A for glucose and hemoglobin detection

| Compound | % (w/w) |
|---|---|
| Water | 53.58 |
| Acetone | 44.15 |
| Tetramethylbenzidine (TMB) | 0.12 |
| Butylated hydroxytoluene (BHT) | 0.01 |
| 6-Methoxyquinoline (6-MXQ) | 0.18 |
| Lepidine | 0.26 |
| Cumene hydroperoxide (CHP) | 0.60 |
| 10% sodium dodecylbenzene- | 1.02 |
| sulfonate (SDBS) w/w | |
| 1M EDTA | 0.07 |
| Glucose oxidase (GOx) | Traces-0.005 |
| Horseradish peroxidase (HRP) | Traces-0.005 |

The composition of the polysaccharide matrices tested are shown at Table 3B:

TABLE 3B
Polysaccharide matrices 3a to 3e

| | Matrix identification and composition (wt %) | | | | |
|---|---|---|---|---|---|
| Pollysaccharide | 3a | 3b | 3c | 3d | 3e |
| Microcrystalline | 30% | 30% | 10% | 30% | 0% |
| cellulose (MCC) | | | | | |
| Pregelatinized | 30% | 57.5% | 67.5% | 55% | 40% |
| starch (PGS) | | | | | |
| Guar gum | 20% | 0% | 0% | 0% | 0% |
| Methyl hydroxyethyl | 20% | 12.5% | 12.5% | 10% | 15% |
| cellulose (Tylose ®) | | | | | |
| Hydroxyethyl | 0% | 0% | 10% | 5% | 5% |
| cellulose | | | | | |
| Powdered fibrous | 0% | 0% | 0% | 0% | 40% |
| cellulose (Arbocel ®, | | | | | |
| grade BWW 40) | | | | | |

For all polysaccharide matrices 3a, 3b, 3c, 3d and 3e: chromogenic granules 3a to 3e were produced by dropwise addition of chromogenic solution of Table 3A directly onto a powder bed of homogeneously prepared polysaccharide matrix 3a, 3b, 3c, 3d or 3e. This resulted in humid, quasi-spherical granules 3a, 3b, 3c, 3d or 3e. The humid granules were immediately transferred onto a strainer to sift out any excess powder matrix and subsequently placed in an oven for drying at 70° C. The granules thereby obtained were then tested for glucose and hemoglobin detection using hemoglobin and glucose solutions.

### Assessment of Granules 3a:

Granules 3a displayed a blue coloration during their production, which disappeared during the drying process;
Granules 3a did not elicit a blue coloration upon addition of demineralized water (negative control).
Granules 3a reacted quickly to produce an almost instantaneously visible, progressively darker blue coloration with increasing glucose concentrations;
Granules 3a also effectively detected hemoglobin solutions and performed similarly to the glucose detection;
Granules 3a did not exhibit the candy effect upon contact with increasingly concentrated glucose solutions;
The disappearance of the candy effect was attributed to the use of methyl hydroxyethyl cellulose, as granules made with a similar polysaccharide matrix that did not include methyl hydroxyethyl cellulose still exhibited the candy effect.

### Assessment of Granules 3b:

Granules 3b performed similarly to granules 3a, for all the points listed above, but were able to detect hemoglobin at a lower concentration than granules 3a. This was attributed to the removal of guar gum from the polysaccharide matrix.

### Assessment of Granules 3c:

Granules 3c performed similarly to granules 3b but the blue coloration was notably more intense than with granules 3b.

### Assessment of Granules 3d:

Granules 3d performed similarly to granules 3b but started to present very minor signs of heterogeneous granule coloration. It was evaluated that lowering the methyl hydroxyethyl cellulose content to lower than 10 wt % of the polysaccharide matrix would reintroduce the candy effect.

### Assessment of Granules 3e:

Granules 3e performed similarly to granules 3a but with a less intense blue coloration. Granules 3e showed that it was possible to obtain granules for combined detection of hemoglobin and glucose without using MCC and by replacing it with a fibrous mechanically treated cellulose (e.g., Arbocel®, grade BWW 40 having an average fibre length of 200 μm, an average fibre thickness of 20 μm and a bulk density between 0.11-0.145 g/mL). However, granules 3e showed a blue coloration when contacted with demineralized water, but not when contacted with cat urine that was free of glucose and hemoglobin.

### Example 4

Granules having the same polysaccharide matrix as in Examples 2 and 3 were manufactured, using:
1) a similar chromogenic solution as outlined at Table 3A but without SDBS or any other anionic surfactant;
2) the chromogenic solution outlined at Table 3A; and
3) a similar chromogenic solution as outlined at Table 3A but replacing SDBS with SDS (sodium dodecyl sulfate).

When the chromogenic solution without any anionic surfactant was used, the granules showed a blue coloration upon contacting a hemoglobin solution and upon contacting a glucose solution. However, the blue coloration typically shifted to army-green about 3 hours after application of the hemoglobin or glucose solutions.

When the SDBS-containing chromogenic solution was used, the granules showed a blue coloration upon contacting a hemoglobin solution and upon contacting a glucose solution. The blue coloration was stable for a longer time and did not shift to army-green until at least 24 hours after application of the hemoglobin or glucose solutions.

When the SDS-containing chromogenic solution was used, the granules showed a light blue coloration that was less intense than when SDBS was used. The sensitivity was therefore lower when using SDS than SDBS.

This Example showed that the use of an arylalkyl sulfonate surfactant such as SDBS stabilized the blue coloration of the granules after contact with a hemoglobin solution or a glucose solution, and allowed for increased sensitivity.

### Example 5

It was observed that with granules 1a, 1b, 2a and 2b, a light blue halo developed on the perimeter of the clump formed upon application of healthy cat urine, when the granules were added to a litter material containing metallic cations (e.g., bentonite contains 2-6% of iron mainly in the form of Fe³⁺). It was hypothesized that this effect may be due to the presence of anionic polymers in the respective polysaccharide matrices. For example, granule 1a contains sodium polyacrylate and granules 1b, 2a and 2b contain carboxymethyl cellulose. Without being bound by theory, one possible explanation could be that upon moistening of the granules when in contact with bentonite particles, the humid medium may allow for the electrostatic migration of Fe³⁺ from bentonite to the granules, resulting in unwanted oxidation of TMB and the unwanted light blue coloration. It was therefore postulated that eliminating all anionic polysaccharides from the polysaccharide matrix may improve or even eliminate this light blue halo at the edge of the clumps.

Granules 3a, 3b, 3c, 3d and 3e are free of anionic polymers (e.g., free of anionic polysaccharides and free of anionic superabsorbent polymers), and indeed do not feature this light blue halo upon application of healthy cat urine.

### Example 6

Experiments were conducted to manufacture and assess granules using one of the chromogenic solutions shown at Table 6A:

TABLE 6A
Chromogenic solutions for glucose and hemoglobin detection

| | Solution 6A | | Solution 6B | | Solution 6C | | Solution 6D | |
|---|---|---|---|---|---|---|---|---|
| | | % | | % | | % | | % |
| Compound | (mg) | w/w | (mg) | w/w | (mg) | w/w | (mg) | w/w |
| Acetone | 22500 | 44.166 | 2250 | 44.080 | 22500 | 44.136 | 22500 | 44.130 |
| TMB | 60 | 0.118 | 160.03 | 0.314 | 90 | 0.177 | 90 | 0.177 |
| BHT | 5.5 | 0.011 | 5.5 | 0.011 | 11 | 0.022 | 16.5 | 0.032 |
| CHP | 306 | 0.601 | 306 | 0.599 | 306 | 0.600 | 306 | 0.600 |
| 6-MXQ | 93.5 | 0.184 | 93.5 | 0.183 | 93.5 | 0.183 | 93.5 | 0.183 |
| Lepidine | 132.5 | 0.260 | 132.5 | 0.260 | 132.5 | 0.260 | 132.5 | 0.260 |
| water | 27300 | 53.589 | 27300 | 53.484 | 27300 | 53.551 | 27300 | 53.544 |
| Ascorbic | 0 | 0 | 0 | 0 | 0 | 0 | 0 | 0 |
| acid | | | | | | | | |
| 1M EDTA | 33.5 | 0.066 | 3.3.5 | 0.066 | 33.5 | 0.066 | 33.5 | 0.066 |
| GOx | 1.6 | 0.003 | 1.6 | 0.003 | 1.6 | 0.003 | 2.4 | 0.005 |
| HRP | 1 | 0.002 | 1 | 0.002 | 1 | 0.002 | 1.5 | 0.003 |
| 10% SDBS | 510 | 1.001 | 510 | 0.999 | 510 | 1.000 | 510 | 1.000 |
| Total | 50943.6 | 100 | 51043.6 | 100 | 50979.1 | 100 | 50985.9 | 100 |

| | Solution 6E | | Solution 6F | | Solution 6G | | Solution 6H | |
|---|---|---|---|---|---|---|---|---|
| | | % | | % | | % | | % |
| Compound | (mg) | w/w | (mg) | w/w | (mg) | w/w | (mg) | w/w |
| Acetone | 22500 | 44.575 | 22500 | 44.574 | 22500 | 44.572 | 22500 | 44.556 |
| TMB | 120 | 0.238 | 120 | 0.238 | 120 | 0.238 | 140 | 0.277 |
| BHT | 11 | 0.022 | 11 | 0.022 | 11 | 0.022 | 11 | 0.022 |
| CHP | 0 | 0 | 0 | 0 | 0 | 0 | 0 | 0 |
| 6-MXQ | 0 | 0 | 0 | 0 | 0 | 0 | 0 | 0 |
| Lepidine | 0 | 0 | 0 | 0 | 0 | 0 | 0 | 0 |
| water | 27300 | 54.084 | 27300 | 54.083 | 27300 | 54.081 | 27300 | 54.061 |
| Ascorbic | 0 | 0 | 0 | 0 | 0 | 0 | 0 | 0 |
| acid | | | | | | | | |
| 1M EDTA | 33.5 | 0.066 | 33.5 | 0.066 | 33.5 | 0.066 | 33.5 | 0.066 |
| GOx | 1.6 | 0.003 | 2.1 | 0.004 | 3.2 | 0.006 | 2.4 | 0.005 |
| HRP | 1 | 0.002 | 1.3 | 0.003 | 2 | 0.004 | 1.5 | 0.003 |
| 10% SDBS | 510 | 1.010 | 510 | 1.010 | 510 | 1.010 | 510 | 1.010 |
| Total | 50477.1 | 100 | 50477.9 | 100 | 50479.7 | 100 | 50498.4 | 100 |

| | | |
|---|---|---|
| Solution 6I | Solution 6J | Solution 6K |

| | | % | | % | | % |
|---|---|---|---|---|---|---|
| Compound | (mg) | w/w | (mg) | w/w | (mg) | w/w |
| Acetone | 22500 | 44.557 | 22500 | 44.509 | 22500 | 44.177 |
| TMB | 140 | 0.277 | 200 | 0.396 | 160 | 0.314 |
| BHT | 11 | 0.022 | 5.5 | 0.011 | 5.5 | 0.011 |
| CHP | 0 | 0 | 0 | 0 | 3.6 | 0.601 |
| 6-MXQ | 0 | 0 | 0 | 0 | 47 | 0.092 |
| Lepidine | 0 | 0 | 0 | 0 | 66 | 0.130 |
| water | 27300 | 54.062 | 27300 | 54.004 | 27300 | 53.601 |
| Ascorbic | 0 | 0 | 0 | 0 | 2 | 0.004 |
| acid | | | | | | |
| 1M EDTA | 33.5 | 0.066 | 33.5 | 0.066 | 33.5 | 0.066 |
| GOx | 1.6 | 0.003 | 1.6 | 0.003 | 1.1 | 0.002 |
| HRP | 1.5 | 0.003 | 1 | 0.002 | 0.7 | 0.001 |
| 10% SDBS | 510 | 1.010 | 510 | 1.009 | 510 | 1.001 |
| Total | 50497.6 | 100 | 50551.6 | 100 | 50931.8 | 100 |

The composition of the polysaccharide matrices tested are shown at Tables 6B, 6C and 6D:

TABLE 6B
Polysaccharide matrices 6.1 to 6.16 (% by weight in the polysaccharide powder)

| Granule # | 6.1 | 6.2 | 6.3 | 6.4 | 6.5 | 6.6 | 6.7 | 6.8 | 6.9 | 6.10 | 6.11 | 6.12 | 6.13 | 6.14 | 6.15 | 6.16 |
|---|---|---|---|---|---|---|---|---|---|---|---|---|---|---|---|---|
| MCC 101 | 30 | 30 | 35 | 40 | 45 | 50 | 32.5 | 32.5 | 32.5 | 32.5 | | | | | | |
| (50 μm mean | | | | | | | | | | | | | | | | |
| particle size) | | | | | | | | | | | | | | | | |
| MCC 102 | | | | | | | | | | | 32.5 | | | | | |
| (100 μm mean | | | | | | | | | | | | | | | | |
| particle size) | | | | | | | | | | | | | | | | |
| Arbocel ™ | | | | | | | | | | | | 32.5 | | | 32.5 | 32.5 |
| Vitacel ™ 601 | | | | | | | | | | | | | 32.5 | | | |
| Vitacel ™ 611 | | | | | | | | | | | | | | 32.5 | | |
| Paygel ™ | 55 | 57.5 | 52.5 | 47.5 | 42.5 | 37.5 | 52.5 | 50 | 47.5 | 47.5 | 47.5 | 47.5 | 47.5 | 47.5 | 47.5 | 47.5 |
| Tylose ™ MH | | 12.5 | 12.5 | 12.5 | 12.5 | 12.5 | 15 | 15 | 15 | 15 | 15 | 15 | 15 | 15 | 15 | |
| 60000 P6 | | | | | | | | | | | | | | | | |
| Tylose MHE | | | | | | | | | | | | | | | | 15 |
| 60000 YP4 | | | | | | | | | | | | | | | | |
| Tylose MHS | | | | | | | | | | | | | | | | |
| 60000 YP4 | | | | | | | | | | | | | | | | |
| Tylose MHS | | | | | | | | | | | | | | | | |
| 300000 P4 | | | | | | | | | | | | | | | | |
| Tylose MH | | | | | | | | | | | | | | | | |
| 300 P2 | | | | | | | | | | | | | | | | |
| HPMC | | | | | | | | | | | | | | | | |
| (Hydroxy- | | | | | | | | | | | | | | | | |
| propylmethyl | | | | | | | | | | | | | | | | |
| cellulose) | | | | | | | | | | | | | | | | |
| Guar G3 | 10 | | | | | | | | | | | | | | | |
| HEC | 5 | | | | | | | 2.5 | 5 | 5 | 5 | 5 | 5 | 5 | 5 | 5 |
| CMC Cekol ™ | | | | | | | | | | | | | | | | |
| 50000 | | | | | | | | | | | | | | | | |
| Total | 100 | 100 | 100 | 100 | 100 | 100 | 100 | 100 | 100 | 100 | 100 | 100 | 100 | 100 | 100 | 100 |
| Chromogenic | 6A | 6A | 6A | 6A | 6.A | 6A | 6A | 6A | 6A | 6A | 6A | 6A | 6A | 6A | 6B | 6B |
| Solution No. | | | | | | | | | | | | | | | | |

TABLE 6C
Polysaccharide matrices 6.17 to 6.32 (% by weight in the polysaccharide powder)

| Granule # | 6.17 | 6.18 | 6.19 | 6.20 | 6.21 | 6.22 | 6.23 | 6.24 | 6.25 | 6.26 | 6.27 | 6.28 | 6.29 | 6.30 | 6.31 | 6.32 |
|---|---|---|---|---|---|---|---|---|---|---|---|---|---|---|---|---|
| MCC 101 | | | | | | | | | | | 40 | 40 | 40 | | | |
| MCC 102 | | | | | | | | | | | | | | | | |
| Arbocel | 32.5 | 32.5 | 32.5 | 32.5 | 32.5 | 32.5 | 32.5 | 45 | 45 | 45 | | | | 40 | 40 | 30 |
| Vitacel 601 | | | | | | | | | | | | | | | | |
| Vitacel 611 | | | | | | | | | | | | | | | | |
| Paygel | 47.5 | 47.5 | 47.5 | 45 | 45 | 42.5 | 42.5 | 45 | 50 | 53 | 45 | 58 | 55 | 45 | 45 | 50 |
| Tylose MH | | | | 15 | | 15 | | | | | 15 | | | 10 | 10 | 15 |
| 60000 P6 | | | | | | | | | | | | | | | | |
| Tylose MHE | | | | | | | | | | | | | | | | |
| 60000 YP4 | | | | | | | | | | | | | | | | |
| Tylose MHS | 15 | | | | | | | | | | | | | | | |
| 60000 YP4 | | | | | | | | | | | | | | | | |
| Tylose MHS | | 15 | | | 15 | | 15 | | | | | | | | | |
| 300000 P4 | | | | | | | | | | | | | | | | |
| Tylose MH | | | 15 | | | | | | | | | | | | | |
| 300 P2 | | | | | | | | | | | | | | | | |
| HPMC | | | | | | | | 10 | 5 | 2 | | | | | | |
| Guar G3 | | | | | | | | | | | | | | | | |
| HEC | 5 | 5 | 5 | 7.5 | 7.5 | 10 | 10 | | | | | | | | 5 | |
| CMC Cekol ™ | | | | | | | | | | | | 2 | 5 | 5 | | 5 |
| 50000 | | | | | | | | | | | | | | | | |
| Total | 100 | 100 | 100 | 100 | 100 | 100 | 100 | 100 | 100 | 100 | 100 | 100 | 100 | 100 | 100 | 100 |
| Chromogenic | 6B | 6B | 6B | 6B | 6B | 6B | 6B | 6C | 6C | 6C | 6D | 6D | 6D | 6D | 6D | 6D |
| Solution No. | | | | | | | | | | | | | | | | |

TABLE 6D
Polysaccharide matrices 6.33 to 6.42 (% by weight in the polysaccharide powder)

| Granule # | 6.33 | 6.34 | 6.35 | 6.36 | 6.37 | 6.38 | 6.39 | 6.40 | 6.41 | 6.42 |
|---|---|---|---|---|---|---|---|---|---|---|
| MCC 101 | 30 | 30 | 30 | | | | | | | |
| MCC 102 | | | | | | | | | | |
| Arbocel | | | | | | | 30 | 30 | 15 | 30 |
| Vitacel 601 | | | | 30 | 30 | 30 | | | | |
| Vitacel 611 | | | | | | | | | | |
| Paygel | 55 | 50 | 52.5 | 55 | 54 | 53 | 50 | 48 | 68 | 53 |
| Tylose MH 60000 P6 | 15 | 15 | 12.5 | 15 | 15 | 15 | 20 | 20 | 15 | 15 |
| Tylose MHE 60000 YP4 | | | | | | | | | | |
| Tylose MHS 60000 YP4 | | | | | | | | | | |
| Tylose MHS 300000 P4 | | | | | | | | | | |
| Tylose MH 300 P2 | | | | | | | | | | |
| HPMC | | | | | | | | | | |
| Guar G3 | | | | | | | | | | |
| HEC | | 5 | 5 | | | | | | | |
| CMC Cekol 50000 | | | | | 1 | 2 | | 2 | 2 | 2 |
| Total | 100 | 100 | 100 | 100 | 100 | 100 | 100 | 100 | 100 | 100 |
| Chromogenic | 6E, | 6E, | 6E, | 6H | 6H | 6H | 6I | 6I | 6J | 6J |
| Solution No. | 6F, | 6F, | 6F, | | | | | | | |
| | 6G | 6G | 6G | | | | | | | |

For all granules 6.1 to 6.42: chromogenic granules were produced by dropwise addition of the chromogenic solution listed in Tables 6B, 6C and 6D directly onto a powder bed of homogeneously prepared polysaccharide matrix. When more than one chromogenic solution is listed in a single Table cell (e.g., E, F, G in Table 6D), it is meant that three separate types of granules were manufactured, each with a single chromogenic solution. This resulted in humid, quasi-spherical granules for each polysaccharide matrix 6.1 to 6.42. The humid granules were immediately transferred onto a strainer to sift out any excess powder matrix and subsequently placed in an oven for drying at 70° C. The granules thereby obtained were then tested for glucose detection using a glucose solution and for hemoglobin detection using a hemoglobin solution.

### Granules 6.1 to 6.3:

Granules 6.1, 6.2 and 6.3 did not exhibit any blue coloration during the production process;
Granules 6.1, 6.2 and 6.3 were reactive to hemoglobin and glucose (i.e., turned blue when wetted with cat urine that included hemoglobin or glucose);
Granules 6.1, 6.2 and 6.3 did not exhibit any false positive results when wetted with cat urine that was free of hemoglobin or glucose;
Granule 6.3 exhibited a slight blue coloration when wetted with demineralized water, but was still functional (i.e., turned to a more pronounced blue) when further wetted with cat urine that included hemoglobin or glucose;
Granules 6.1 and 6.2 did not exhibit a blue coloration when wetted with demineralized water.

### Granules 6.4 to 6.6:

Granules 6.4 to 6.6 exhibited a slight blue coloration during the production process, which did not dissipate after drying;
Granules 6.4 to 6.6 were reactive to hemoglobin and glucose;
Granules 6.4 to 6.6 produced false positive results when wetted with demineralized water or cat urine that included hemoglobin or glucose.

### Granules 6.7 to 6.9:

Granules 6.6 to 6.9 exhibited a slight blue coloration during the production process, which dissipated after drying;
Granules 6.6 to 6.9 were reactive to hemoglobin and glucose;
Increasing HEC in the polysaccharide matrix resulted in a more intense blue coloration for hemoglobin detection;
Granules 6.6 to 6.9 did not produce any false positive result when wetted with cat urine that was free of hemoglobin or glucose.

### Granules 6.10 to 6.14:

Granules 6.10 to 6.14 exhibited a slight blue coloration during the production process, which dissipated after drying;
Granules 6.10 to 6.14 were reactive to hemoglobin and glucose;
Granules 6.10 to 6.14 did not produce any false positive result when wetted with cat urine that was free of hemoglobin or glucose;
Granules 6.12, 6.13 and 6.14 containing mechanically processed cellulose (Arbocel, Vitacel 601, Vitacel 611) resulted in a more intense blue coloration for the detection of hemoglobin and glucose;
With granules 6.10 to 6.14, it was shown that various grades of microcrystalline cellulose (e.g., 50 μm mean particle size or 100 μm mean particle size) and various grades of mechanically processed cellulose (Arbocel, Vitacel 601, Vitacel 611) can be used to produce granules that were reactive to both hemoglobin and glucose.

### Granules 6.15 to 6.19:

Granules 6.15 to 6.19 exhibited a slight blue coloration during the production process, which dissipated after drying;
Granules 6.15 to 6.19 were reactive to hemoglobin and glucose;
Granules 6.15 to 6.19 did not produce any false positive result when wetted with cat urine that was free of hemoglobin or glucose;
With granules 6.15 to 6.19, it was shown that various grades of MHEC can be used to produce granules that were reactive to both hemoglobin and glucose.

### Granules 6.20 to 6.23:

Granules 6.20 to 6.23 exhibited a slight blue coloration during the production process, which dissipated after drying;
Granules 6.20 to 6.23 were reactive to hemoglobin and glucose;
Granules 6.20 to 6.23 did not produce any false positive result when wetted with cat urine that was free of hemoglobin or glucose;
With granules 6.20 to 6.23, it was shown that increasing HEC content still allowed producing granules that were reactive to both hemoglobin and glucose.

### Granules 6.24 to 6.31:

Granules 6.24 to 6.31 were reactive to hemoglobin and glucose;
Granules 6.24 to 6.31 produced false positive results when wetted with demineralized water or cat urine that included hemoglobin or glucose.

### Granules 6.32:

Granules 6.32 were reactive to hemoglobin and glucose, produced a homogeneous blue coloration and did not produce false positive results upon wetting with demineralized water or cat urine that included hemoglobin or glucose. The blue coloration did not start fading or turning to green for at least 24 hours.

### Granules 6.33 to 6.42:

Granules 6.33 to 6.42 were manufactured with chromogenic solutions for glucose detection only;
Granules 6.33 to 6.42 were reactive to glucose;
reactivity to glucose increased with increased enzyme concentrations when wetted with both aqueous glucose and glucose-supplemented urine;
granules with CMC generally produced a more pronounced coloration upon contacting a glucose solution;
Granules 6.33 to 6.42 did not produce any false positive result when wetted with cat urine that was free of glucose, or when wetted with demineralized water;

### Example 7

Experiments were conducted to manufacture and assess granules using one of the chromogenic solutions shown at Table 7A:

TABLE 7A
Chromogenic solutions for glucose and hemoglobin detection

| | | Solution 7B | Solution 7C | Solution 7D | Solution 7E |
|---|---|---|---|---|---|
| | Solution 7A | SDS | SDBS | SDBS | SDBS |
| | no surfactant | surfactant | surfactant | surfactant | surfactant |
| Compound | % w/w | % w/w | % w/w | % w/w | % w/w |
| Acetone | 44.624 | 44.177 | 44.399 | 44.177 | 43.957 |
| TMB | 0.317 | 0.314 | 0.316 | 0.314 | 0.313 |
| BHT | 0.011 | 0.011 | 0.011 | 0.011 | 0.011 |
| CHP | 0.607 | 0.601 | 0.604 | 0.601 | 0.598 |
| 6-MXQ | 0.093 | 0.092 | 0.093 | 0.092 | 0.092 |
| Lepidine | 0.131 | 0.130 | 0.130 | 0.130 | 0.129 |
| water | 54.143 | 53.601 | 53.871 | 53.601 | 53.334 |
| Ascorbic | 0.004 | 0.004 | 0.004 | 0.004 | 0.004 |
| acid | | | | | |
| 1M EDTA | 0.066 | 0.066 | 0.066 | 0.066 | 0.065 |
| GOx | 0.002 | 0.002 | 0.002 | 0.002 | 0.002 |
| HRP | 0.001 | 0.001 | 0.001 | 0.001 | 0.001 |
| Surfactant | 0 | 1.001 | 0.503 | 1.001 | 1.495 |
| Total | 100 | 100 | 100 | 100 | 100 |

| | Solution 7F | Solution 7G | Solution 7H | Solution 7I |
|---|---|---|---|---|
| | SDBS | SDBS | SDBS | SDBS |
| | surfactant | surfactant | surfactant | surfactant |
| | % w/w | % w/w | % w/w | % w/w |
| Acetone | 43.730 | 43.284 | 42.835 | 42.391 |
| TMB | 0.311 | 0.308 | 0.305 | 0.302 |
| BHT | 0.011 | 0.0110.011 | 0.010 | 0.010 |
| CHP | 0.595 | 0.589 | 0.583 | 0.577 |
| 6-MXQ | 0.091 | 0.090 | 0.089 | 0.089 |
| Lepidine | 0.128 | 0.127 | 0.126 | 0.124 |
| water | 53.059 | 52.518 | 51.973 | 51.435 |
| Ascorbic | 0.004 | 0.004 | 0.004 | 0.004 |
| acid | | | | |
| 1M EDTA | 0.065 | 0.064 | 0.064 | 0.063 |
| GOx | 0.002 | 0.002 | 0.002 | 0.002 |
| HRP | 0.001 | 0.001 | 0.001 | 0.001 |
| Surfactant | 2.002 | 3.001 | 4.007 | 5.002 |
| Total | 100 | 100 | 100 | 100 |

The surfactants in Table 7A are added as a 10% w/w aqueous solution. The surfactant are therefore present at a concentration of 0 wt %, 0.10 wt %, 0.05 wt %, 0.10 wt %, 0.15 wt %, 0.20 wt %, 0.30 wt %, 0.40 wt % and 0.50 wt %, in chromogenic solutions 7A, 7B, 7C, 7D, 7E, 7F, 7G and 7I, respectively, based on the total weight of the respective chromogenic solution.

The composition of the polysaccharide matrix tested is shown at Table 7B:

TABLE 7B
Polysaccharide matrix 7

| | composition |
|---|---|
| | (wt %) |
| Pollysaccharide | 7 |
| Unfunctionalized, mechanically treated | 30 |
| cellulose (Arbocel ™ BWW-40) | |
| Pregelatinized starch (Paygel ™ 290) | 50 |
| Methyl hydroxyethyl cellulose | 15 |
| (Tylose ™ MHS 60000 YP4) | |
| Carboxymethyl cellulose (Cekol ™ 50000P) | 5 |

It was recently reported in the literature (Li, Meng; Huang, Xiang-Rong; Guo, Yi; Shang, Ya-Zhuo; Liu, Hong-Lai (2017), Chinese Chemical Letters, hereby incorporated by reference in its entirety) that sodium dodecyl sulfate (SDS), an anionic surfactant, may help stabilize the blue coloration of the positively charged TMB complex, in solution. It was surprisingly found that using an aryl alkyl sulfonate (e.g., sodium dodecylbenzene sulfonate (SDBS)) instead of the alkyl sulfonate SDS gave a more intense blue color, thereby increasing the sensitivity of the hemoglobin and glucose detection.

Various SDBS concentrations were tested. It was found that granules produced with a surfactant concentration ranging from 0.05 wt % to 0.50 wt % based on a total weight of the chromogenic solution displayed a marked increase in coloration intensity, which did not fade before at least 24 hours, and which did not shift to green. Using an SDBS concentration in this range allowed reaching a level of detection of about 10 mg/dl for glucose and about 30 RBC/μL for hemoglobin. Using SDS allowed reaching a similar level of detection, but the color shifted to green a few hours after turning blue.

### Example 8

Experiments were conducted to manufacture and assess granules using one of the chromogenic solutions shown at Table 8A:

TABLE 8A
Chromogenic solutions for glucose and hemoglobin detection

| | | Solution 8B | Solution 8C | Solution 8D |
|---|---|---|---|---|
| | Solution 8A | SDS | SDBS | SHS |
| | no surfactant | surfactant | surfactant | surfactant |
| Compound | % w/w | % w/w | % w/w | % w/w |
| Acetone | 44.624 | 44.177 | 44.177 | 43.756 |
| TMB | 0.317 | 0.314 | 0.314 | 0.311 |
| BHT | 0.011 | 0.011 | 0.011 | 0.011 |
| CHP | 0.607 | 0.601 | 0.601 | 0.595 |
| 6-MXQ | 0.093 | 0.092 | 0.092 | 0.091 |
| Lepidine | 0.131 | 0.130 | 0.130 | 0.128 |
| water | 54.143 | 53.601 | 53.601 | 53.090 |
| Ascorbic | 0.004 | 0.004 | 0.004 | 0.004 |
| acid | | | | |
| 1M EDTA | 0.066 | 0.066 | 0.066 | 0.065 |
| GOx | 0.002 | 0.002 | 0.002 | 0.002 |
| HRP | 0.001 | 0.001 | 0.001 | 0.001 |
| Surfactant | 0 | 1.001 | 1.001 | 1.945 |
| Total | 100 | 100 | 100 | 100 |

| | Solution 8E | Solution 8F | Solution 8G |
|---|---|---|---|
| | SBS | TS | Empigen ™ BB |
| | surfactant | surfactant | surfactant |
| | % w/w | % w/w | % w/w |
| Acetone | 43.756 | 44.177 | 44.470 |
| TMB | 0.314 | 0.314 | 0.316 |
| BHT | 0.011 | 0.011 | 0.011 |
| CHP | 0.601 | 0.601 | 0.605 |
| 6-MXQ | 0.092 | 0.092 | 0.093 |
| Lepidine | 0.130 | 0.130 | 0.130 |
| water | 53.601 | 53.601 | 53.956 |
| Ascorbic | 0.004 | 0.004 | 0.004 |
| acid | | | |
| 1M EDTA | 0.066 | 0.066 | 0.066 |
| GOx | 0.002 | 0.002 | 0.002 |
| HRP | 0.001 | 0.001 | 0.001 |
| Surfactant | 1.001 | 1.001 | 0.345 |
| Total | 100 | 100 | 100 |

The surfactants in Table 8A are added as a 10% aqueous solution. The surfactant are therefore present at a concentration of 0 wt %, 0.10 wt %, 0.10 wt %, 0.19 wt %, 0.10 wt %, 0.10 wt % and 0.034 wt %, in chromogenic solutions 8A, 8B, 8C, 8D, 8E, 8F and 7G, respectively, based on the total weight of the respective chromogenic solution.

The composition of the polysaccharide matrix tested is shown at Table 8B:

TABLE 8B
Polysaccharide matrix 8

| | composition |
|---|---|
| | (wt %) |
| Pollysaccharide | 8 |
| Unfunctionalized, mechanically treated | 30 |
| cellulose (Arbocel ™ BWW-40) | |
| Pregelatinized starch (Paygel ™ 290) | 50 |
| Methyl hydroxyethyl cellulose | 15 |
| (Tylose ™ MHS 60000 YP4) | |
| Carboxymethyl cellulose (Cekol ™ 50000P) | 5 |

An additional surfactant (CTAB) was tested. Because of CTAB's low solubility, granules were produced with a chromogenic solution devoid of surfactant, and the surfactant was added to the polysaccharide matrix which was supplemented with either 0.27 wt % or 0.69 wt % CTAB (w/w).

The surfactants tested were the following:
SDS: Sodium dodecyl sulfate;
SDBS: Sodium dodecylbenzene sulfonate;
SHS: Sodium hexanesulfonate;
SBS: Sodium benzene sulfonate;
TS: Sodium toluene-4-sulfonate;
CTAB: Hexadecyltrimethylammonium bromide; and
Epigen BB: N-(Alkyl C10-C16)-N,N-dimethylglycine betaine.

### Hemoglobin Detection:

Granules produced with any of the anionic surfactants remained reactive to hemoglobin and produced unequivocal blue coloration upon contact with a hemoglobin solution. However, granules produced with SDBS were the most reactive and sensitive, detecting hemoglobin at concentrations as low as 30 RBC/μL, exhibited a more intense coloration, shifted the least towards greener colors, all while resisting fading at LOD levels (about 30 RBC/μL for hemoglobin). SDS was almost as effective at stabilizing granule coloration at low hemoglobin concentration than SDBS but was unable to resist a color shift to greener colors. Finally, SHS, SBS and TS did not allow for a stable granule coloration at low hemoglobin concentrations and the blue color shifted toward green after a few hours.

Granules produced with cationic surfactant CTAB negatively affected granule performance. Although reactive, granules containing CTAB lacked sensitivity, showed a shift to green after a few hours and the color faded significantly after a few hours-more so than with granules devoid of surfactant.

Granules produced with amphoteric surfactant Empigen BB produced an unequivocal blue coloration at concentrations as low as 30 RBC/μL. However, the coloration at LOD concentrations began fading 1 hour after being wetted with biomarker solution. A shift to green was also observed after a few hours.

### Glucose Detection:

Granules produced with any of the anionic surfactants were reactive and produced an unequivocal blue coloration upon contact with a glucose solution. Despite all granules produced with anionic molecules/surfactants being reactive, sensitive, and resisting fading in time, only those containing SDBS exhibited the most intense coloration at LOD levels (10 mg/dl for glucose an) of glucose and shifted the least towards green over time.

Granules produced with CTAB were reactive and produced an unequivocal blue coloration upon contact with a glucose solution. Granules containing CTAB performed similarly to those produced with any of the anionic molecules, except for SDBS; Granules containing CTAB were as reactive and sensitive as granules containing SDBS, produced an unequivocal blue coloration on the granules even at LOD levels of glucose, which resisted fading in time but were unable to withstand the color shift to greener hues after a few hours.

Granules produced with Empigen BB performed similarly to those containing SDBS, rapidly producing an unequivocal blue color at LOD levels of Glucose. However, granules produced with Empigen BB exhibited a shift to green after a few hours.

1.-36. (canceled)

37. A chromogenic absorbent material for hemoglobin and/or glucose detection in an animal excretion, the chromogenic absorbent material comprising:
a benzidine-type compound;
at least one of:
an organic hydroperoxide, for catalyzing the oxidation of the benzidine-type compound in the presence of hemoglobin; and
a catalytic system comprising:
a first enzyme which is an oxidoreductase, for in situ generation of hydrogen peroxide in the presence of glucose; and
a second enzyme which is a peroxidase, a pseudoperoxidase or a combination thereof, for catalyzing the oxidation of the benzidine-type compound upon in situ generation of the hydrogen peroxide;
a salt of alkylbenzene sulfonate; and
a polysaccharide matrix comprising a chemically or mechanically processed non-functionalized cellulose and pregelatinized starch (PGS).

38. The chromogenic absorbent material of claim 37, wherein the salt of alkylbenzene sulfonate is a salt of dodecylbenzene sulfonate.

39. The chromogenic absorbent material of claim 37, wherein the polysaccharide matrix comprises:
from about 10 wt % to about 32.5 wt % chemically or mechanically processed non-functionalized cellulose;
from about 25 wt % to about 70 wt % PGS;
from 0 wt % to about 20 wt % guar gum;
from 0 wt % to about 25 wt % methyl hydroxyethyl cellulose (MHEC);
from 0 wt % to about 15 wt % hydroxyethyl cellulose (HEC); and
from 0 wt % to about 10 wt % carboxymethyl cellulose (CMC).

40. The chromogenic absorbent material of claim 39, wherein the polysaccharide matrix comprises:
from about 10 wt % to about 32.5 wt % chemically or mechanically processed non-functionalized cellulose;
from about 25 wt % to about 70 wt % PGS;
from about 10 wt % to about 20 wt % MHEC; and
from about 2.5 wt % to about 7.5 wt % CMC.

41. The chromogenic absorbent material of claim 39, wherein the polysaccharide matrix comprises:
from about 25 wt % to about 32.5 wt % chemically or mechanically processed non-functionalized cellulose;
from about 40 wt % to about 60 wt % PGS;
from about 10 wt % to about 20 wt % MHEC; and
from about 2.5 wt % to about 7.5 wt % CMC.

42. The chromogenic absorbent material of claim 37, wherein the chemically or mechanically processed non-functionalized cellulose is microcrystalline cellulose (MCC), fibrous cellulose, or a combination thereof.

43. The chromogenic absorbent material of claim 42, wherein the microcrystalline cellulose has an average particle size by laser diffraction between about 25 μm and about 200 μm, and wherein the fibrous cellulose has an average fiber length of up to about 220 μm.

44. The chromogenic absorbent material of claim 37, wherein the chemically or mechanically processed non-functionalized cellulose is a mechanically processed non-functionalized cellulose having an average particle size by laser diffraction between about 50 μm and about 80 μm, and a bulk density between about 0.20 g/mL and about 0.35 g/mL.

45. The chromogenic absorbent material of claim 38, wherein the salt of dodecylbenzene sulfonate is sodium dodecylbenzene sulfonate, ammonium dodecylbenzene sulfonate, triethylamine dodecylbenzene sulfonate, potassium dodecylbenzene sulfonate or a combination thereof.

46. The chromogenic absorbent material of claim 37, wherein the first enzyme is glucose oxidase (GOx) and the second enzyme is horseradish peroxidase (HRP).

47. The chromogenic absorbent material of claim 37, wherein the benzidine-type compound comprises 3,3′,5,5′-tetramethylbenzidine.

48. The chromogenic absorbent material of claim 37, wherein the organic hydroperoxide is cumene hydroperoxide, diisopropylbenzene dihydroperoxide, or a combination thereof.

49. The chromogenic absorbent material of claim 37, further comprising a buffering agent, a stabilizer, a metal scavenger, a color enhancer, or a combination thereof.

50. The chromogenic absorbent material of claim 37, having a density of about 0.20 g/cm³ to about 0.39 g/cm³.

51. The chromogenic absorbent material of claim 37, material having an effective porosity of about 0.5 mL/g to about 2.0 mL/g.

52. The chromogenic absorbent material of claim 37, which has pores having an equivalent diameter greater than about 20 μm.

53. The chromogenic absorbent material of claim 37, having a free swelling capacity greater than about 900%.

54. The chromogenic absorbent material of claim 37, having a pore density greater than about 20%.

55. The chromogenic absorbent material of claim 37, wherein:
the chemically or mechanically processed non-functionalized cellulose is microcrystalline cellulose (MCC), fibrous cellulose, or a combination thereof;
the salt of alkylbenzene sulfonate is sodium dodecylbenzene sulfonate, ammonium dodecylbenzene sulfonate, triethylamine dodecylbenzene sulfonate, potassium dodecylbenzene sulfonate, or a combination thereof;
the first enzyme is glucose oxidase (GOx) and the second enzyme is horseradish peroxidase (HRP);
the benzidine-type compound comprises 3,3′,5,5′-tetramethylbenzidine; and
the organic hydroperoxide is cumene hydroperoxide, diisopropylbenzene dihydroperoxide, or a combination thereof.

56. The chromogenic absorbent material of claim 37, which is for hemoglobin and glucose detection and comprises the organic hydroperoxide and the catalytic system.
